(19) United States
(12) Patent Application Publication
(10) Pub. No.: US 2018/0224456 A1
(43) Pub. Date: Aug. 9, 2018

(54) METHOD FOR DETERMINING A SUBJECT'S PROBABILITY TO SUFFER FROM PANCREATIC CANCER

(71) Applicant: Reccan Diagnostics AB, Lund (SE)

(72) Inventor: Roland Andersson, Lund (SE)

(21) Appl. No.: 15/506,425

(22) PCT Filed: Aug. 26, 2015
(86) PCT No.: PCT/EP2015/069557

§ 371 Date: Feb. 24, 2017

(30) Foreign Application Priority Data
Aug. 26, 2014 (SE) 1450091-3

(51) Int. Cl. G01N 33/574

(52) U.S. Cl. CPC G01N 33/57438; G16H 50/30

(57) ABSTRACT

A method for determining a subject's probability to suffer from pancreatic cancer, wherein the level of Platelet Glycoprotein V (GP5), or a peptide fragment thereof is used as a biomarker. An increased level of GP5, or a peptide fragment thereof, is indicative for an increased probability to suffer from pancreatic cancer.

### TECHNICAL FIELD

The present invention relates to methods for determination of probability of presence of pancreatic cancer.

### BACKGROUND

Pancreatic cancer often presents clinically at an advanced stage because symptoms appear late in the course of the disease and patients are therefore not diagnosed until after development of distant metastasis [1]. The survival rate is the lowest among human solid tumors, with a median survival of only 6 months [2]. Pancreatic cancer is classified as resectable (stages I-II; 10-20%), locally advanced (stage III; 30%) or distant metastatic (stage IV; 60%) [3]. Patients with resectable cancers can potentially be cured by complete surgical removal [4]. Therefore, new, non-invasive approaches are crucial in order to improve early detection. In terms of clinical utility, serum is an attractive source of biomarkers due to the low invasiveness and easy sample processing. The biomarker discovery is of utmost importance since currently only CA 19-9, a carbohydrate antigen, is available as a serum tumor marker for pancreatic cancer. CA 19-9 has properties that are insufficient both in terms of sensitivity as well as specificity, for early diagnosis [5]. Due to low positive predictive value and the fact that benign pancreatic disorders and all forms of biliary obstruction can increase CA 19-9 levels, CA 19-9 is not recommended for use as a screening test for pancreatic cancer.

Clinical suitability of a biomarker depends on several factors, such as availability, simplicity or robustness of analysis techniques for which the biomarker offers high enough sensitivity and specificity for successful determination during routine clinical practice.

Recent advances in mass spectrometry techniques have enabled the investigation of protein expression profiles in complex protein mixtures, and the identification and quantification of disease-perturbed proteins. Traditionally, two-dimensional gel electrophoresis (2-DE) has been the main method used for mass spectrometry-based proteomic profiling [6]. However, 2-DE is limited by factors such as being experimentally laborious, and being difficult to perform reproducibly and consequently challenging for high-throughput analysis [7]. As an alternative to the 2-DE approach, ‘bottom-up’ shotgun proteomics has emerged. The shotgun approach uses a proteolytic enzyme such as trypsin to generate peptides that can be analyzed with LC-MS/MS [8-10]. However, given the complexity of the serum and plasma proteome only a few studies have investigated the use of shotgun proteomics for the discovery of pancreatic cancer biomarkers in blood [11]. A reason for this is the need for rigorous epidemiological projects or clinical trials for determining accuracy, reliability, interpretability, and feasibility of a biomarker. This has to be established with consideration to variables such as age, gender, intraindividual variation, tissue localization and persistence of the biomarker.

Hence, improved methods based on the analysis of relevant biomarkers in samples from patients are needed for improved diagnosis of pancreatic cancer.

### SUMMARY

It is an object of the present invention, considering the disadvantages mentioned above, to provide a method which enables a complementary or stand-alone assessment of the probability that a subject, e.g., a patient, is suffering from pancreatic cancer in comparison to a reference subject, e.g., a healthy individual.

According to a first aspect of the invention, there is provided a method for determining a subject's probability to suffer from pancreatic cancer comprising the steps of: (i) Providing a first sample from a subject whose probability to suffer from pancreatic cancer is to be determined, and determining the level of Platelet Glycoprotein V (GP5), or a peptide fragment thereof, in the first sample; (ii) providing a second sample from a reference subject not suffering from pancreatic cancer, and determining the level of Platelet Glycoprotein V (GP5), or a peptide fragment thereof, in the second sample and (iii) comparing the level of Platelet Glycoprotein V (GP5), or a peptide fragment thereof, in said first and second sample. The steps (i) and (ii) can be carried out in any order. An increased level of GP5, or a peptide fragment thereof, in the first sample is indicative for an increased probability to suffer from pancreatic cancer.

In some forms, a serum concentration of GP5, or a peptide fragment thereof, in the first sample at least 30% higher than of the second sample is indicative for an increased probability to suffer from pancreatic cancer. In some forms, a concentration of GP5 1.978 μg/L in said first sample is indicative for an increased probability to suffer from pancreatic cancer.

In some forms, steps (i) and (ii) also comprises determining the level of at least one other protein or polypeptide in said first and second sample, said one protein or polypeptide being selected from the group consisting of CEA (Carcinoembryonic antigen), tumor marker CA 242, TAG-72 (Tumor-associated glycoprotein 72), HNRNPCL1, CA19-9, G7d, KAT2B, KIF20B, SMC1B and/or SPAG5 proteins. Also, step (iii) further comprises comparing the level of said at least one other protein or polypeptide in said first and second sample, and wherein an increased level of GP5, or a peptide fragment thereof, and said protein or polypeptide is indicative for an increased probability to suffer from pancreatic cancer. In some forms, the at least one protein or polypeptide is selected from the group consisting of Heterogeneous nuclear ribonucleoprotein C-like 1 (HNRNPCL1) and carbohydrate antigen 19-9 (CA19-9), and an increased level of GP5, or a peptide fragment thereof, and Heterogeneous nuclear ribonucleoprotein C-like 1 (HNRNPCL1) and/or carbohydrate antigen 19-9 (CA19-9) in the first sample compared to the second sample is indicative for an increased probability to suffer from pancreatic cancer. In some forms, the at least one protein or polypeptide is carbohydrate antigen 19-9 (CA19-9), and wherein a value of 2.729 or more for 0.562417*log (level GP5 in μg/L)+0.400120*log (level CA19-9 in μg/L) is indicative for an increased probability to suffer from pancreatic cancer.

In some forms, step (i) and (ii) comprises treating said samples or a derivative thereof with a protease. Said protease selectively cleaves at least a part of the peptide bonds of the comprising proteins and polypeptides thereof at the carboxylic acid side of lysine and arginine residues, which provides a plurality of polypeptide fragments. The level is determined of at least one polypeptide fragment among the plurality of polypeptide fragments from the group consisting of SeqIDNo30, SeqIDNo31, SeqIDNo32 in said samples, wherein the fragment levels are directly correlating to the initial level of Platelet Glycoprotein V (GP5) in said samples.

According to another aspect of the invention, there is provided a method for determining a subject's probability to suffer from pancreatic cancer, comprising the steps of (i) providing a sample from a subject whose probability to suffer from pancreatic cancer is to be determined and determining the level of Platelet Glycoprotein V (GP5), or a peptide fragment thereof, in the sample; and (ii) comparing the level of Platelet Glycoprotein V (GP5), or a peptide fragment thereof, with a reference value determined based on the level of Platelet Glycoprotein V (GP5), or a peptide fragment thereof, in samples from subjects known to suffer from pancreatic cancer and the level of Platelet Glycoprotein V (GP5), or a peptide fragment thereof, in samples from healthy subjects. A level of Platelet Glycoprotein V (GP5), or a peptide fragment thereof, above the reference value in said sample is indicative for an increased probability to suffer from pancreatic cancer.

In some forms, the reference value is 1.978 μg/L. In some forms, a serum concentration of GP5, or a peptide fragment thereof, of more than 1.978 μg/ml, but less than 4.5 μg/L in said sample is indicative for an increased probability to suffer from pancreatic cancer stage I-II. In some forms, a serum concentration of GP5, or a peptide fragment thereof, of more than 4.5 μg/L in said sample is indicative for an increased probability to suffer from pancreatic cancer stage III-IV. In some forms, the reference value is a combination of a level of Platelet Glycoprotein V (GP5), or a peptide fragment thereof, and a level of carbohydrate antigen 19-9 (CA19-9), and a value of 2.729 or more for 0.562417*log (level GP5 in μg/L)+0.400120*log (level CA19-9 in μg/L) is indicative for an increased probability to suffer from pancreatic cancer.

According to a third aspect of the invention, Platelet Glycoprotein V (GP5), or a peptide fragment thereof, is used as a biomarker for pancreatic cancer. In some forms, also CA19.9 and/or HNRNPCL1 are used as co-biomarker(s).

According to a fourth aspect of the invention, an element binding to Platelet Glycoprotein V (GP5), or a peptide fragment thereof, is used in detecting Platelet Glycoprotein V (GP5), or a peptide fragment thereof, as biomarker indicative for pancreatic cancer, in a sample from a subject. Is some forms, said element binding to Platelet Glycoprotein V (GP5), or a peptide fragment thereof, is an antibody or a fragment thereof. In some forms, said element is used in an ELISA (enzyme-linked immunosorbent assay) or EIA (enzyme immunoassay).

According to a fifth aspect of the invention, a kit comprising means for measuring the level of Platelet Glycoprotein V (GP5), or a peptide fragment thereof, in a sample from a subject is provided.

Further advantageous features of the invention are defined in the dependent claims. In addition, advantageous features of the invention are elaborated in embodiments disclosed herein.

### BRIEF DESCRIPTION OF THE DRAWINGS

These and other aspects, features and advantages of which the invention is capable of will be apparent and elucidated from the following description of the present invention, reference being made to the accompanying drawings, in which

FIG. 1 is schematic of an experimental pipeline for high definition mass spectrometry (HDMS^{E}). UPLC, UltraPerformance Chromatography,

FIG. 2 is a software visualization of raw HDMS^{E} data overlayed tripled injections,

FIG. 3 is a heat map diagram with two-way unsupervised hierarchical clustering of proteins and serum samples. Each row represents a protein and each column represents a sample. The protein clustering tree is shown on the left, and the sample clustering tree appears at the top. The scale shown in the map illustrates the relative expression level of a protein across all samples. This analysis identified 134 differentially expressed proteins (p<0.0009). There was clustering of 40 proteins up-regulated in pancreatic cancer as compared to patients with benign pancreatic disease and healthy controls (Table 3).

FIG. 4 is a graph showing a principal component analysis on the differentially expressed proteins between pancreatic cancer, benign pancreatic disease and healthy controls,

FIG. 5 is a gene ontology classification of proteins identified in the serum samples, showing molecular function in a clockwork fashion starting in a clockwork order,

FIG. 6 shows a diagram with GP5 abundance for the diagnosis of pancreatic cancer, including cancer stages I-II and an ROC curve showing the range of sensitivity and specificity for cancer prediction that is obtained by varying the threshold value of GP5 abundance,

FIG. 7 shows a diagram with GP5 and CA19.9 abundance for the diagnosis of pancreatic cancer, including cancer stages I-II and an ROC curve showing the range of sensitivity and specificity for cancer prediction that is obtained by varying the threshold value of GP5 abundance,

FIG. 8 shows a diagram with GP5 abundance for the differentiation between pancreatic cancer stages I-II and an ROC curve showing the range of sensitivity and specificity for cancer prediction that is obtained by varying the threshold value of GP5 abundance.

### DETAILED DESCRIPTION

Embodiments of the present invention will be described in more detail below with reference to the accompanying figures in order for those skilled in the art to be able to carry out the invention. The invention may, however, be embodied in many different forms and should not be construed as limited to the embodiments set forth herein. Rather, these embodiments are provided so that this disclosure will be thorough and complete, and will fully convey the scope of the invention to those skilled in the art. The embodiments do not limit the invention, but the invention is only limited by the appended patent claims. Furthermore, the terminology used in the detailed description of the particular embodiments illustrated in the accompanying drawings is not intended to be limiting of the invention.

To reach beyond the limitations of conventional mass spectrometry, the use of high definition mass spectrometry (HDMS^{E}) can provide the extra dimension of high-efficiency ion mobility separation to achieve deeper proteome coverage [12]. In the findings underlying the present invention, shotgun proteomics with HDMS^{E} was used to examine serum proteins from patients with resectable pancreatic cancer as well as patients with benign pancreatic disease and healthy controls. The identified serum proteome differences were subjected to protein network analysis for investigation of protein-protein interactions.

Pancreatic cancer is commonly detected at advanced stages when the tumor is no longer amenable to surgical resection. Therefore, finding biomarkers for early stage disease is urgent. It was shown that high definition mass spectrometry (HDMS^{E}) can be used to identify serum protein alterations associated with early stage pancreatic cancer, representing potential biomarkers for early stage pancreatic cancer. Serum samples from pancreatic cancer patients diagnosed with operable tumors as well as patients with benign pancreatic disease and healthy controls were analyzed. The SYNAPT G2-Si platform was used in a data-independent manner coupled with ion mobility. The dilution of the samples with a yeast alcohol dehydrogenase tryptic digest of known concentration allowed the estimated amounts of each identified protein to be calculated. When injected in triplicates the MS spectra clustered tightly and showed highly reproducible separation demonstrating that the number of replicates could be reduced to two and hence reduce analytical time.

A global protein expression comparison of the three study groups, i) pancreatic cancer, ii) benign pancreatic disease and iii) healthy controls, was made using label-free quantification and bioinformatic analyses. Two-way unsupervised hierarchical clustering with 134 differentially expressed proteins (p<0.0009) successfully classified pancreatic cancer patients from the controls, and identified 40 proteins that showed a significant up-regulation in the pancreatic cancer group, thus representing potential biomarkers for early stage pancreatic cancer.

This discrimination reliability was further confirmed by principal component analysis (PCA). The differentially expressed candidates were aligned with protein network analyses and linked to biological pathways related to pancreatic tumorigenesis. Pancreatic disease link associations could be made to p53, the most frequently altered tumor suppressor in pancreatic cancer. These pancreatic cancer study candidates may provide new avenues of research for a non-invasive blood based diagnosis for pancreatic tumor stratification.

As already stated, early pancreatic cancer detection and treatment is hampered by the lack of accurate diagnostic biomarkers. To reduce the mortality of pancreatic cancer patients, detection of cancer at curable stages is the best approach at present. A comprehensive, systematic characterization of serum protein profiles in disease and control specimens from our South Swedish Pancreas Biobank may facilitate development of biomarkers for diagnosis of pancreatic cancer. One important strategy for discovery of pancreatic cancer biomarkers is mass spectrometry-based proteomic analysis of body fluids including blood [11]. However, although serum and plasma are important sources for investigating pancreatic cancer-related biomarkers, the complexity of their proteome is a challenge. In this study, a systematic approach for the discovery of pancreatic cancer biomarkers: (1) dedicated sample preparation in serum, (2) HDMS^{E} for the identification of differentially expressed proteins with label-free quantification using an internal standard, (3) hierarchical clustering and (4) PCA was attempted.

In this feasibility study, it was demonstrated that HDMS^{E} can be used to discover potential biomarkers in sera from pancreatic cancer patients. The platform provides resolution in three dimensions and allows for high peak capacity analyses maximizing protein identification whilst retaining label-free quantification capabilities. Relative quantification analysis of the three conditions was performed using a label free approach. Hierarchical clustering and PCA of the data showed a clear differentiation between the pancreatic cancer and control phenotypes.

According to one embodiment, a subject's probability to suffer from pancreatic cancer relative a reference subject may comprise a first step of providing a first sample being representative of the subject's proteome. The first sample may be a blood, plasma, or tissue sample. A second step may involve treatment of the first sample or a derivative thereof with a protease. The protease will typically selectively cleave at least a part of the peptide bonds of the proteins and polypeptides present in the first sample at the carboxylic acid side of lysine and arginine residues, to provide a plurality of polypeptide fragments. A derivative of the first sample may be the proteins and polypeptides remaining after treatments, such as e.g. purification to remove proteins not being related to pancreatic cancer or cleavage of S—S bonds to provide linear protein sequences, of the first sample. An example of a suitable protease is trypsin, such as e.g. porcine trypsin being rendered resistant to proteolytic digestion by modification by reductive methylation. A third step may be the determination of the presence or level of at least one polypeptide fragment among the plurality of polypeptide fragments obtained in the second step. Several such polypeptide fragments may typically be quantified to provide a better basis for comparison with a reference sample, e.g. a sample from a reference subject, in order to minimize the risk of false positive or negative results. A second sample being representative of the reference subject's proteome may be provided as a fourth step. Preferably, the second sample may be of the same type as the first sample. As a fifth step, the second sample, or a derivative thereof, may be treated under the same conditions, preferably by employment of the same protocol, as the first sample during the second step. Any derivative of the second sample may preferably be obtained according to the same protocol as the provision of the derivative of the first sample. The presence or level of the same polypeptide fragments as determined in the resulting composition after protease treatment of the first sample or derivative thereof may then be determined after the corresponding treatment of the second sample, as a sixth step. As a final seventh step, the level or presence of each relevant polypeptide fragment obtained from the first and second sample are compared with each other. A higher level in a sample, derived from the first sample, of a polypeptide fragment resulting from peptidase assisted cleavage of an endogenous protein or polypeptide which is increased in the presence of pancreatic cancer, as compared to the corresponding sample derived from the second sample, indicates a higher probability of the subject to suffer from pancreatic cancer as compared to the reference subject's probability of suffering of the same. Accordingly, a lower level in a sample, derived from the first sample, of a polypeptide fragment resulting from peptidase assisted cleavage of an endogenous protein or polypeptide which is decreased in the presence of pancreatic cancer, as compared to the corresponding sample derived from the second sample, indicates a higher probability of the subject to suffer from pancreatic cancer as compared to the reference subject's probability of suffering of the same.

According to one embodiment, the endogenous proteins or polypeptides, which increase or decrease in the presence of pancreatic cancer as compared to a healthy subject as described herein, may be quantitatively determined by LC-MS, LC-MS/MS, gel-electrophoresis or by employment of a detectable moiety adapted to selectively bind to at least one such endogenous protein or polypeptide.

According to one embodiment, the polypeptide fragments obtained by treatment with trypsin of the endogenous proteins or polypeptides, which increase or decrease in the presence of pancreatic cancer as compared to a healthy subject as described herein, may be quantitatively determined by LC-MS, LC-MS/MS, gel-electrophoresis or by employment of a detectable moiety adapted to selectively bind to at least one such polypeptide fragment.

The study led to the identification of a 40-protein panel that seemingly distinguishes pancreatic cancer from benign and healthy controls. To better understand potential underlying mechanisms of importance in pancreatic cancer, a series of protein network analyses was performed using the differentially regulated proteins that were identified in the experiments. Among this protein set, examples of proteins whose abundance were found to be the increased in pancreatic cancer included GP5, HNRNPC, G7d, KAT2B, KIF20B, SMC1B and SPAG5. These proteins are proteins present at low concentrations in the blood stream, thus revealing the successful potential of our strategy to identify low-abundant candidate cancer biomarkers.

According to one embodiment, the significant increase in level of one or more of the following peptides or polypeptides, or polypeptide fragments (within parenthesis) when having been treated with trypsin, in a proteome sample of a subject, in comparison to the corresponding sample of healthy individual, may be indicative of the presence of pancreatic cancer in the subject: SeqIDNo118 (SeqIDNo3, SeqIDNo4, SeqIDNo5, SeqIDNo6, SeqIDNo7, SeqIDNo8, SeqIDNo9, SeqIDNo10), SeqIDNo120 (SeqIDNo15, SeqIDNo16, SeqIDNo17, SeqIDNo18), SeqIDNo122 (SeqIDNo27, SeqIDNo28), SeqIDNo123 (SeqIDNo29), SeqIDNo124 (SeqIDNo30, SeqIDNo31, SeqIDNo32), SeqIDNo126 (SeqIDNo41A, SeqIDNo42, SeqIDNo43, SeqIDNo44, SeqIDNo45, SeqIDNo46, SeqIDNo47, SeqIDNo48, SeqIDNo49), SeqIDNo128 (SeqIDNo69, SeqIDNo70, SeqIDNo71, SeqIDNo72, SeqIDNo73, SeqIDNo74, SeqIDNo75, SeqIDNo76, SeqIDNo77, SeqIDNo78, SeqIDNo79, SeqIDNo80, SeqIDNo81), SeqIDNo132 (SeqIDNo85, SeqIDNo86), SeqIDNo134 (SeqIDNo88, SeqIDNo89), SeqIDNo135 (SeqIDNo90), SeqIDNo137 (SeqIDNo95, SeqIDNo96), SeqIDNo140 (SeqIDNo99, SeqIDNo100, SeqIDNo101), SeqIDNo143 (SeqIDNo104); SeqIDNo144 (SeqIDNo105) and SeqIDNo145 (SeqIDNo106, SeqIDNo107, SeqIDNo108, SeqIDNo109, SeqIDNo110, SeqIDNo111).

According to one embodiment, the significant decrease in level of one or more of the following peptides or polypeptides, or polypeptide fragments (within parenthesis) when having been treated with trypsin, in a proteome sample of a subject, in comparison to the corresponding sample of healthy individual, may be indicative of the presence of pancreatic cancer in the subject: SeqIDNo117 (SeqIDNo1, SeqIDNo2), SeqIDNo119 (SeqIDNo11, SeqIDNo12, SeqIDNo13, SeqIDNo14), SeqIDNo121 (SeqIDNo19, SeqIDNo20, SeqIDNo21, SeqIDNo22, SeqIDNo23, SeqIDNo24, SeqIDNo25, SeqIDNo26), SeqIDNo125 (SeqIDNo33, SeqIDNo34, SeqIDNo35, SeqIDNo36, SeqIDNo37, SeqIDNo38, SeqIDNo39, SeqIDNo40), SeqIDNo127 (SeqIDNo50, SeqIDNo51, SeqIDNo52, SeqIDNo53, SeqIDNo54, SeqIDNo55, SeqIDNo56, SeqIDNo57, SeqIDNo58, SeqIDNo59, SeqIDNo60, SeqIDNo61, SeqIDNo62, SeqIDNo63, SeqIDNo64, SeqIDNo65, SeqIDNo66, SeqIDNo67, SeqIDNo68), SeqIDNo129 (SeqIDNo82), SeqIDNo130 (SeqIDNo83), SeqIDNo131 (SeqIDNo84), SeqIDNo133 (SeqIDNo87), SeqIDNo136 (SeqIDNo91, SeqIDNo92, SeqIDNo93, SeqIDNo94), SeqIDNo138 (SeqIDNo97), SeqIDNo139 (SeqIDNo98), SeqIDNo141 (SeqIDNo102), SeqIDNo142 (SeqIDNo103), SeqIDNo146 (SeqIDNo112, SeqIDNo113), SeqIDNo147 (SeqIDNo114) and SeqIDNo148 (SeqIDNo115, SeqIDNo116).

Differentially expressed candidates, as can be seen in table 3, with link associations to p53, the most frequently altered tumor suppressor in pancreatic cancer could also be made for BAZ2A, CDK13, DAPK1, DST, EXOSC3, INHBE, KAT2B, KIF20B, SMC1B and SPAG5.

Thus, according to one embodiment, the significant decrease in level of the following peptide or polypeptide, or polypeptide fragments (within parenthesis) when having been treated with trypsin, in a proteome sample of a subject, in comparison to the corresponding sample of healthy individual, may be indicative of the presence of pancreatic cancer in the subject: SeqIDNo117 (SeqIDNo1, SeqIDNo2).

According to one embodiment, the significant increase in level of the following peptide or polypeptide, or polypeptide fragment (within parenthesis) when having been treated with trypsin, in a proteome sample of a subject, in comparison to the corresponding sample of healthy individual, may be indicative of the presence of pancreatic cancer in the subject: SeqIDNo123 (SeqIDNo29).

According to one embodiment, the significant decrease in level of the following peptide or polypeptide, or polypeptide fragments (within parenthesis) when having been treated with trypsin, in a proteome sample of a subject, in comparison to the corresponding sample of healthy individual, may be indicative of the presence of pancreatic cancer in the subject: SeqIDNo119 (SeqIDNo11, SeqIDNo12, SeqIDNo13, SeqIDNo14).

The recent advances in proteomic methods have enabled the systematic characterization of complex proteomes and identification of differentially expressed proteins in cells, tissue and biofluids. To find possible cancer biomarkers, great care must be taken to define the clinical application and to select relevant specimens for proteomic analysis [13]. When analyzing serum or plasma by proteomic methods there are several sources of variability that may occur. One of the most important factors leading to false discovery begins with the choice of adequate controls. Changes in inflammation and acute phase proteins often occur in malignant conditions including pancreatic cancer [14]. These changes may reflect the underlying chronic condition (e.g. chronic pancreatitis) in contrast to cancer-specific changes. Therefore nonspecific changes in serum or plasma need to be differentiated from potentially specific biomarkers. This is why in addition to healthy control specimens, specimens from patients with chronic pancreatitis and other benign pancreatic diseases also were included to adequately identify disease-perturbed proteins.

Further, comparison with healthy control specimens and specimens from patients with chronic pancreatitis allows for determining a threshold value to distinguish between healthy and diseased specimens with sufficient sensitivity and specificity. Methods for such determinations are known in the art. As an example, Receiver Operating Characteristic (ROC) curve analysis may be used.

Clinical suitability of a biomarker depends on several factors, such as availability, simplicity or robustness of analysis techniques. Furthermore, a biomarker must offer high enough sensitivity (i.e. true positive rate) and specificity (i.e. true negative rate) for the analysis technique for successful determination during routine clinical practice.

Solid-phase enzyme-linked immunosorbent assays (ELISA) is a proven method both for general biomedical research and as a diagnostic tool. It allows detection of biological molecules at very low concentrations and quantities. It utilizes the concept of an antigen binding to a specific antibody and the method commonly immobilizes the antigen from the fluid phase into 96 well plates. The antigen binds to a specific antibody, which is itself subsequently detected by a secondary, enzyme-coupled antibody. The high sensitivity of ELISA comes from using an enzyme as a reporting group, and a chromogenic substrate for the enzyme yields a visible color change or fluorescence, indicating the presence of the antigen. Quantitative or qualitative measures can be assessed based on such colorimetric reading. By ELISA antibody quantification can be done at microgram or even nanogram levels. The high specificity of ELISA is due to the selectivity of the antibody or antigen. ELISA also adds the advantage of not requiring radioisotopes (radioactive substances) or a costly radiation counter (a radiation-counting apparatus), such as in radioimmune assay (RIA) tests, making it a readily available technique in most standard laboratory environments.

A cohort of biomarkers containing of GP5, HNRNPC, G7d, KAT2B, KIF20B, SMC1B and SPAG5 proteins was selected for determining their clinical suitability using the ELISA method.

ELISA quantification is a well-known method to the skilled person. As an example, for GP5 ELISA quantification, rabbit polyclonal antibodies raised against recombinant GP5 are pre-coated in microtiter plates. A fixed amount of blood serum samples is added and incubated in the plates. After incubation, the liquid is exchanged for a solution containing detection antibodies, conjugated to biotin. After further incubation, the wells are washed and a solution containing Horse radish peroxidase (HRP) is added. HRP is a glycoprotein which produces a coloured, fluorimetric, or luminescent derivative of the labeled molecule when incubated with a proper substrate, such as 3,3′, 5,5′-Tetramethylbenzidine (TMB). TMB acts as a hydrogen donor for the reduction of hydrogen peroxide to water by HRP, resulting in a diimine of a blue colour which can be read on a spectrophotometer at a wavelength of 650 nm. After incubation, TMB substrate is added. If there is GP5 in the sample, wells containing biomarker, biotin conjugated antibody and the enzyme conjugated avidin will exhibit a color change which correlates to the amount of GP5 present in the blood serum sample. In this way, the level of Platelet Glycoprotein V (GP5) in the subject's sample can be determined.

In one embodiment, an element binding to Platelet Glycoprotein V (GP5), or a peptide fragment thereof, is used in detecting Platelet Glycoprotein V (GP5), or a peptide fragment thereof, as biomarker indicative for pancreatic cancer, in a sample from a subject. The element may be used in an ELISA (enzyme-linked immunosorbent assay) or EIA (enzyme immunoassay). As recognized by the skilled person, the element binding to Platelet Glycoprotein V (GP5), or a peptide fragment thereof, may an antibody or a fragment thereof. Useful fragments of antibodies may be selected from the group consisting of F(ab′)₂, Fab′, Fab, ScFv di-scFv, sdAb fragments. The element may be modified or linked to functional groups, such as biotin, streptavidin or avidin for binding of the element, or enzymes, such as horseradish peroxidase (HRP), alkaline phosphatase (AP), β-galactosidase, acetylcholinesterase and catalase, for use as a reporting group together with a corresponding substrate.

In another embodiment a kit comprising means for measuring the level of Platelet Glycoprotein V (GP5), or a peptide fragment thereof, in a sample from a subject, is provided. Such as kit is useful in practicing the various methods disclosed herein. For ELISA, such a kit may comprise a capture antibody, preferably coated or immobilized on a microplate, binding to a first antigenic site of Platelet Glycoprotein V (GP5), or a peptide fragment thereof. Further, a detecting antibody binding to a secondary antigenic site of Platelet Glycoprotein V (GP5), or a peptide fragment thereof is typically part of the kit. The first and second antigenic binding sites may be identical, in the case where multiple identical antigenic binding sites exist. Also, an enzyme-linked secondary antibody binding to said detecting antibody and substrate being converted by said enzyme to a detectable form. Further, the kit may comprises a detecting antibody binding to Platelet Glycoprotein V (GP5), an enzyme-linked secondary antibody binding to the detecting antibody, and a substrate being converted by said enzyme to detectable form. Furthermore, the kit may also comprises a capture antibody binding to Platelet Glycoprotein V (GP5) and being bound to surface, such as a microplate.

In direct ELISA, the antigen (here Platelet Glycoprotein V (GP5)) is adsorbed directly to a plastic surface (i.e microplate well). A protein, such as bovine serum albumin, is thereafter added in abundance to block all the other binding sites. The enzyme-antibody complex is then applied and bound to the antigen. After excess antibodies are washed away, the enzyme's substrate can be applied for ELISA analysis. This enables the use of a single enzyme linked antibody. In one embodiment, the kit thus comprises a primary enzyme-linked antibody binding to Platelet Glycoprotein V (GP5), and substrate being converted by said enzyme to detectable form.

All selected biomarkers, i.e. GP5, HNRNPC, G7d, KAT2B, KIF20B, SMC1B and SPAG5, fulfill several criteria for suitability, such as being released in the blood stream and being upregulated/downregulated in pancreatic cancer. Out of the cohort, it was found that GP5 (Human platelet glycoprotein V) had the highest clinical suitability using the robust and sensitive ELISA technique. The results are summarized in Table 4, where GP5 clearly stands out as the best pancreatic biomarker using ELISA method of the cohort.

GP5 is a part of the Ib-V-IX system of surface glycoproteins that constitute the receptor for von Willebrand factor (VWF; MIM 613160) and mediate the adhesion of platelets to injured vascular surfaces in the arterial circulation, a critical initiating event in hemostasis. Thrombin as well as diverse metalloproteases cleave GP5, generating peptide fragments that are easily quantified in serum using enzyme-linked immunosorbent assay (ELISA). Moreover, elevated plasma levels of peptide platelet GP5 are linked to development of thrombosis which represents one of the major complication in patients with unresectable pancreatic cancer.

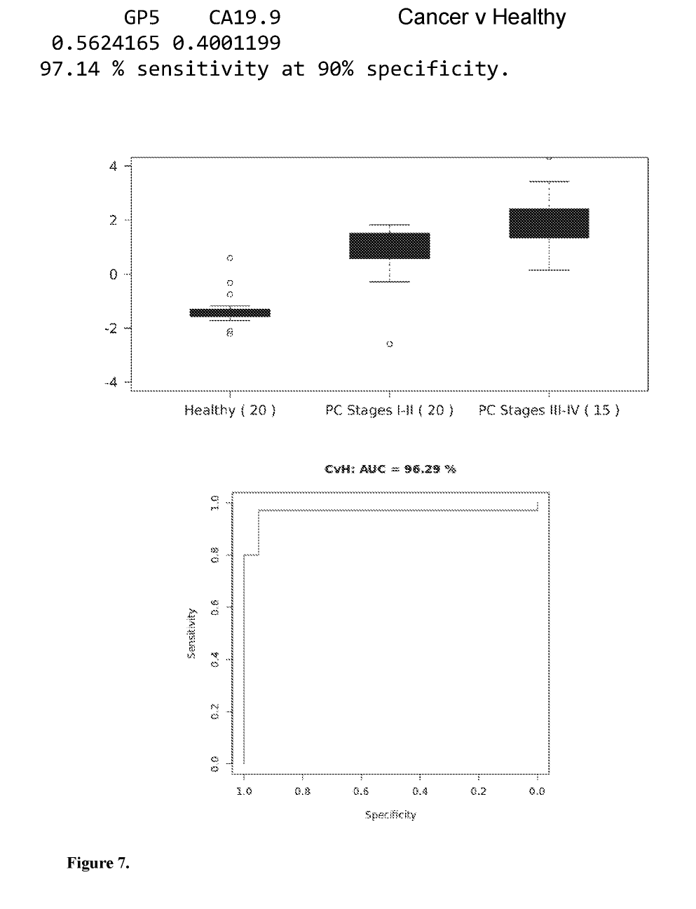

GP5 abundance for the whole ELISA patient group of Table 1, as verified by ELISA method, is specified in Table 5. GP5 provides both high sensitivity and specificity for determining a subject's probability to suffer from pancreatic cancer, which is shown in more detail in FIG. 7. It is also shown that healthy patients are clustered together in a well defined group in relation to pancreatic cancer patients. The AUC (area under the curve) for discriminating pancreatic cancer from healthy controls reached 91%, with a sensitivity of 77% at 90% specificity.

One embodiment of the invention thus relates to use of Platelet Glycoprotein V (GP5), or a peptide fragment thereof, as a biomarker for pancreatic cancer.

Further, one embodiment of the invention relates to a method for determining a subject's probability to suffer from pancreatic cancer, by using GP5 as a biomarker. This is achieved by comparing the level of Platelet Glycoprotein V (GP5), or a peptide fragment thereof, in a sample relative the level of GP5, or a peptide fragment thereof, in a reference sample from a reference subject not suffering from pancreatic cancer. Further, the level of Platelet Glycoprotein V (GP5), or a peptide fragment thereof, in the subject's sample may be compared to a reference value representative for the level of Glycoprotein V (GP5), or a peptide fragment thereof, in samples from subjects not suffering from pancreatic cancer. An increased level of GP5, or a peptide fragment thereof, is indicative for increased probability to suffer from pancreatic cancer. Further, another embodiment relates to a method for identifying a subject suffering from pancreatic cancer, e.g. diagnosing, or assisting in diagnosing, pancreatic cancer. Such a method is similar to the method of determining a subject's probability to suffer from pancreatic cancer, as an increased level of GP5, or a peptide fragment thereof, is indicative for increased probability to suffer from pancreatic cancer. Thus, a subject with increased level of GP5, or a peptide fragment thereof, may be diagnosed with pancreatic cancer with such a method.

According to an embodiment, determining a subject's probability to suffer from pancreatic cancer relates to stratifying a subject relative a healthy reference subject or a reference value, as disclosed herein below, into a first group with no increased probability to suffer from pancreatic cancer or into a second group with increased probability to suffer from pancreatic cancer. Further, as elaborated herein below, the actual level of GP5, or a peptide fragment thereof, may be used to stratifying the subject into a first group of stage I-II pancreatic cancer, or into a second group with group of stage II-IV pancreatic cancer, as discussed further herein below. According to another embodiment, determining a subject's probability to suffer from pancreatic cancer relates to a method for assisting in diagnosing, or for diagnosing, pancreatic cancer in a subject. An increased level of GP5, or a peptide fragment thereof, is indicative for the subject suffering from pancreatic cancer.

This may be achieved by taking a sample of the subject's proteome, such as a blood, plasma, or tissue sample. Preferably the sample is a blood sample, such as a plasma or serum sample. The level of Platelet Glycoprotein V (GP5), or a peptide fragment thereof, in the sample may then be determined using a method, for example ELISA, MS or LC-MS, as described in materials and methods. Similarly, a sample (one or several) may be taken in a similar manner from a reference subject (one or several) not suffering from pancreatic cancer. The level of Platelet Glycoprotein V (GP5), or a peptide fragment thereof, in the reference sample is determined in a similar manner. As several reference samples may be used the reference level determined may be an average value. By comparing the determined level of Platelet Glycoprotein V (GP5), or a peptide fragment thereof, for the subject and the reference subject, the probability that the subject suffers from pancreatic cancer can be determined, as increased level of GP5, or a peptide fragment thereof, is indicative for increased probability to suffer from pancreatic cancer. For subjects shown to have an increased probability to suffer from pancreatic cancer, further examination, such as second-level abdominal imaging, may then be performed to confirm or rule out pancreatic cancer. Thus, GP5 may be used as a biomarker in screening for pancreatic cancer to allow for early detection of it.

Human GP5 has an extracellular topological domain, a transmembrane domain and cytoplasmic domain and an n-terminal signal peptide which can be cleaved at different sites. Furthermore, there are known mutations for GP5, some which are linked to known bleeding disorders.

In one embodiment of the invention, the Platelet Glycoprotein V (GP5) comprises a polypeptide sequence which is at least 90% homologous, such as at least 95% homologous, or even homologous to SeqIDNo124, or wherein the peptide fragment thereof is at least 90% homologous, preferably at least 95% homologous or even homologous, to the corresponding part of SeqIDNo124.

According to one embodiment, a GP5 concentration in a subject which is at least 30% higher, at least 40% higher, or even at least 50% higher, than the GP5 concentration of healthy controls is indicative for discriminating pancreatic cancer in a subject. Thus, a subject with a peripheral blood level of GP5 at least 30% higher, at least 40% higher, or even at least 50% higher, than peripheral blood level of GP5 in healthy individuals is indicative of the subject having pancreatic cancer. Using higher value will improve the sensitivity, but decrease the specificity, as appreciated by the skilled person.

According to an embodiment, the reference level of Platelet Glycoprotein V (GP5) is an average value of at least two, typical several (i.e. 3, 4, 5, 10, 15, 20, 25, 50 or more), previously determined values from at least two, typical several (i.e. 3, 4, 5, 10, 15, 20, 25, 50 or more), different reference subjects. As already explained, the level may be determined using a method such as ELISA, MS or LC-MS. By comparing the determined level of Platelet Glycoprotein V (GP5) for the subject and the average value of several previously determined values, representing the reference subject, the probability that the subject suffers from pancreatic cancer may be determined.

In one embodiment, the subject and the reference subject is the same person, but from whom the sample used as reference sample was collected at a time when the person didn't suffer from pancreatic cancer. By comparing the determined level of Platelet Glycoprotein V (GP5) for the subject to the sample collected from the subject at a time when the person didn't suffer from pancreatic cancer, representing the reference subject, the probability that the subject suffers from pancreatic cancer can be determined.

Reliability or repeatability of a biomarker is crucial for clinical suitability. Biomarker trials may indicate the clinical sensitivity and specificity of a biomarker. The sensitivity measures the proportion of positives that are correctly identified (i.e. correctly identified sick patients) while the specificity measures the proportion of negatives that are correctly identified (i.e. correctly identified healthy patients). In an ideal situation the biomarker has a clear predictive value but in many cases one needs to be established through clinical trials and statistical analysis. When choosing a cut-off value for determining a disease that offers high sensitivity, this often comes at a price of lowering specificity, i.e. getting a higher rate of false positive.

For pancreatic cancer, it is of importance to minimize false negative diagnoses, since disease symptoms are often detected at a late stage while the cancer may progress quickly and can be treated more effectively at early stages. However, it is also of importance to minimize false positives, since a positive test will have to be followed up by diagnosis methods such as computed tomography (CT scan) and endoscopic ultrasound (EUS) ultrasonography or fine needle aspiration biopsy, which will both draw on medical resources and producing anxiety for the patient.

The use of receiver-operator characteristic curves can provide the tools necessary to determine the best choice in terms of sensitivity and false-positive rates, as can be seen in FIGS. 7 to 9. Using statistical analysis, a suitable cut-off value for determining pancreatic cancer in a patient using ELISA method was determined to be 1.978 μg/L in samples from peripheral blood. However, also higher and lower cut-off values may be used, depending on the desired sensitivity and specificity.

According to an embodiment, a measured GP5 serum level of 1.978 μg/L or more is indicative for discriminating pancreatic cancer from healthy controls. Thus, a subject with a peripheral blood level of GP5 of less than 1.978 μg/L is indicative of the subject not having pancreatic cancer. Similarly, a subject with a peripheral blood level of GP5 1.978 μg/L or more is indicative of the subject having pancreatic cancer, or at least an increased probability to suffer from pancreatic cancer.

According to a further embodiment, a method for determining a subject's probability to suffer from pancreatic cancer is provided. In such a method the level of Platelet Glycoprotein V (GP5), or a peptide fragment thereof, in a sample from a subject whose probability to suffer from pancreatic cancer is to be determined is determined. The level of Platelet Glycoprotein V (GP5), or a peptide fragment thereof, in the sample is then compared with a reference value. A serum concentration above the reference value in said first sample is indicative for an increased probability to suffer from pancreatic cancer. As described a suitable reference value may be determined based on the level of Platelet Glycoprotein V (GP5) in samples from subjects known to suffer from pancreatic cancer and the level of Platelet Glycoprotein V (GP5) in samples from healthy subjects. Further, the level of Platelet Glycoprotein V (GP5) in samples from subjects from benign pancreatic diseases may also be used in determining a suitable reference value. In order to be suitable, i.e. to provide specificity and selectivity, the reference value is typically somewhat higher than the average level of Platelet Glycoprotein V (GP5) in samples from healthy subjects. According to an embodiment, the reference value is 1.978 μg/L.

Conventional biomarker PDAC diagnosis using CA19-9 (carbohydrate antigen 19-9), an epitope of sialylated Lewis blood group antigen, is known to have several drawbacks. In patients who lack the Lewis, which is about 10% of the Caucasian population, CA19-9 is not expressed creating false negatives. False positive expression may also occur in benign pathological conditions, such as obstructive jaundice. However, by combining GP5 with CA19.9 for pancreatic cancer screening, the sensitivity and specificity of the determination can be increased. Furthermore, individual biomarker shortcomings, such as described for CA19.9 above, will not be as severe to the determination when the determination relies on GP5 and Ca19.9.

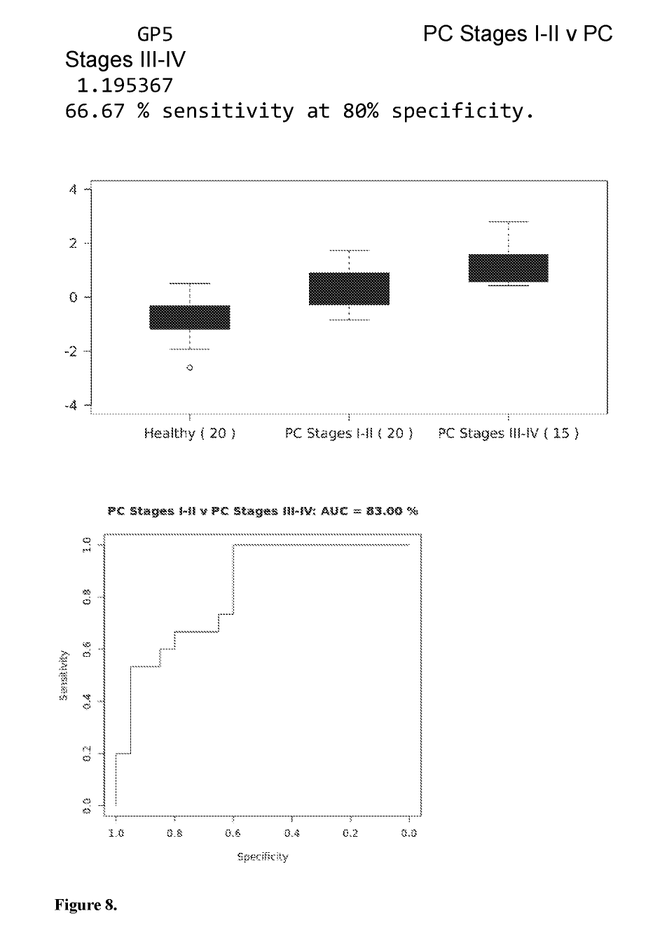

FIG. 8 shows the advantages of GP5 analysis together with CA19.9 in determining a subject's probability to suffer from pancreatic cancer. The AUC for discriminating pancreatic cancer from healthy controls reached 96%, with a sensitivity of 97% at 90% specificity. Using GP5 in combination with CA19.9 will not only provide an improved prediction, it will also greatly reduce the risk of a false positives or negatives compared to conventional treatment, thus reducing the risk of delayed treatment or maltreatment.

According to an embodiment, not only the level of GP5, but also of CA19.9 is determined. An increased level of GP5, or a peptide fragment thereof, and carbohydrate antigen 19-9 (CA19-9) is indicative for an increased probability to suffer from pancreatic cancer. In embodiments wherein the levels of GP5 and CA19.9 are to be compared to a reference value, a value of 2.729 or more for 0.562417*log (level GP5 in μg/L)+0.400120*log (level CA19-9 in μg/L) may be indicative for an increased probability to suffer from pancreatic cancer.

Out of the cohort of biomarkers determined for their clinical suitability using ELISA method, Heterogeneous nuclear ribonucleoprotein C-like 1 (HNRNPCL1) was also found promising. As shown in Table 4, using GP5 together with HNRNPCL1 in determining a subject's probability to suffer from pancreatic cancer was shown provide an improved prediction. Heterogeneous nuclear ribonucleoproteins (hnRNPs) are complexes of RNA and protein present in the cell nucleus. The proteins bound to a pre-mRNA molecule signals that the pre-mRNA is not yet fully processed and ready for export to the cytoplasm. Most RNA-binding proteins in the nucleus exist as heterogeneous ribonucleoprotein particles. After splicing, where pre-mRNA introns are removed and exons are joined, the proteins remain bound to spliced introns which are then targeted for degradation. Elevated HNRNPC expression is known to be play a role in hereditary vitamin D resistance. Furthermore, HNRNPC has been shown to interact with Growth factor receptor-bound protein 2 (Grb2), an adaptor protein involved in signal transduction/cell communication.

In one embodiment, GP5 is thus determined for the subject together with Heterogeneous nuclear ribonucleoprotein C-like 1 (HNRNPCL1). By comparing the determined level of GP5 and HNRNPCL1 to the average value of several previously determined values, representing the reference subject, the probability that the subject suffers from pancreatic cancer can be determined, as increased levels are indicative for an increased probability to suffer from pancreatic cancer.

According to an embodiment, not only the level of GP5, but also of Heterogeneous nuclear ribonucleoprotein C-like 1 (HNRNPCL1) is determined. An increased level of GP5, or a peptide fragment thereof, and HNRNPCL1 is indicative for an increased probability to suffer from pancreatic cancer.

GP5 can be used together with the other up-regulated proteins in pancreatic cancer of Table 3, in particular together with G7d, KAT2B, KIF20B, SMC1B and/or SPAG5 proteins. In one embodiment, GP5 is determined for the subject together with a protein or polypeptide selected from the group consisting of CEA (Carcinoembryonic antigen), tumor marker CA 242, TAG-72 (Tumor-associated glycoprotein 72), HNRNPCL1, CA19-9, G7d, KAT2B, KIF20B, SMC1B and SPAG5 proteins. By comparing the determined level of GP5 together with the selected protein to the average value of several previously determined values, representing the reference subject, the probability that the subject suffers from pancreatic cancer can be determined, as increased levels are indicative for an increased probability to suffer from pancreatic cancer.

Table 4 also shows the results of the combination of GP5 together with both Heterogeneous nuclear ribonucleoprotein C-like 1 (HNRNPCL1) and carbohydrate antigen 19-9 (CA19-9). This combination shows extremely good results with a 100% sensitivity and 100% specificity with a AUC (area under the curve) for discriminating pancreatic cancer from healthy controls of 100%. Thus, greatly reducing the risk of false positives or negatives compared to conventional treatment.

In one embodiment, GP5 is thus determined for the subject together with carbohydrate antigen 19-9 (CA19-9) and Heterogeneous nuclear ribonucleoprotein C-like 1 (HNRNPCL1). By comparing the determined level of GP5 together with Ca19.9 and HNRNPCL1 to the average value of several previously determined values, representing the reference subject, the probability that the subject suffers from pancreatic cancer can be determined, as increased levels are indicative for an increased probability to suffer from pancreatic cancer.

GP5 can be used together with other existing biomarkers, such as CEA (Carcinoembryonic antigen), tumor marker CA 242, TAG-72 (Tumor-associated glycoprotein 72) and circulating nucleosomes connected to pancreatic cancer, such as including nucleosome associated methylated DNA (5 methylcytosine) and histone modifications H2AK119Ub, H3K4Me2, as well as histone sequence variants H2AZ and mH2A1.1. In one embodiment, GP5 is determined for the subject together with a biomarker selected from the group consisting of CEA (Carcinoembryonic antigen), tumor marker CA 242, TAG-72 (Tumor-associated glycoprotein 72) and circulating nucleosomes connected to pancreatic cancer. By comparing the determined level of GP5 together with the selected biomarker to the average value of several previously determined values, representing the reference subject, the probability that the subject suffers from pancreatic cancer can be determined as increased levels are indicative for an increased probability to suffer from pancreatic cancer.

To help decide a treatment plan for pancreatic cancer patients, pancreatic tumors are divided into categories from I to IV, which indicates the severity of the disease and whether surgical removal seems possible, as this is currently the only cure for this cancer. When the disease is still in an early stage (stages I and II), surgical resection of the tumor is normally possible. For stages III and IV a tumor may be inoperable and either neoadjuvant therapy to downstage the tumor to allow subsequent resection should be considered or allow for other treatments such as chemotherapy and radiotherapy to extend life or improve its quality. Despite improvements in preoperative imaging modalities, many potentially resectable tumors are found to be unresectable at laparotomy. Thus, it is of high importance to determine the category of the pancreatic tumor as early as possible.

As can be seen in FIG. 9, GP5 serum levels can not only be used to identify subjects with increased probability to suffer from pancreatic cancer, but also to differentiate between pancreatic cancer patients undergoing surgical exploration for potentially resectable disease. The AUC for the discrimination of pancreatic cancer Stages I-II from Stages III-IV reached 83%, with a sensitivity of 66.6% at 90% specificity. Thus, GP5 levels may aid in preoperatively determining resectability of pancreatic cancer in order to avoid unnecessary explorative laparotomy. In one embodiment the serum concentration of GP5 is used to determine if a pancreatic cancer subject is suffering from pancreatic cancer stage I-II or pancreatic cancer stage III-IV.

As already explained, a serum concentration of GP5 >1.978 ug/ml is indicative for an increased probability to suffer from pancreatic cancer. According to an embodiment, a concentration of GP5 of more than 1.978 ug/ml, but less than 4.5 μg/L is indicative for an increased probability to suffer from pancreatic cancer stage I-II, whereas a serum concentration of GP5 of 4.5 ug/ml or more, indicative for an increased probability to suffer from pancreatic cancer stage III-IV. Similarly, GP5 serum levels can be used during perioperational treatment of pancreatic cancer, as an indicator of the success of surgical removal of a pancreatic tumor, or for monitoring post-resection recurrence and disease progression. If the GP5 level in a subject decreases after resection of the pancreatic cancer, this is indicative of successful surgical removal of a pancreatic tumor or part of a tumor. If the GP5 level in a subject increases after resection of the pancreatic cancer, this is indicative of post-resection recurrence. Thus, the GP5 level in a subject can be used to monitor disease progression during the perioperational phase of pancreatic cancer.

In one embodiment, the subject is in the perioperational phase after surgical removal of pancreatic cancer, a first sample is provided from the subject before surgical removal of pancreatic cancer and a second sample is provided during the perioperational phase after surgical removal of pancreatic cancer. Possibly, the said first and second samples can be taken from the subject at different times during the perioperational phase after surgical removal of pancreatic cancer. By comparing the first and second samples, GP5 serum levels can be tracked over time to determine the subject's disease progression in the perioperational phase.

A decrease in concentration of GP5 over time during the perioperative phase after surgical removal of pancreatic cancer, which can be determined by comparing the GP5 level in the second sample to the first sample, is indicative of successful surgical removal or reduction in mass of pancreatic cancer tumor, according to one embodiment. An increase in GP5 concentration over time in a subject in the perioperative phase after surgical removal of pancreatic cancer, which can be determined by comparing the GP5 level in the second sample to the first sample, is indicative of post-resection pancreatic cancer recurrence and pancreatic cancer disease progression.

Material and Methods

Serum biofluids included in this study were prospectively sampled from patients with pancreatic cancer, benign pancreatic disease, as well as healthy controls. The study patients were undergoing treatment at the Department of Surgery, Skåne University Hospital, Lund, Sweden, between March 2012 and June 2014. Peripheral blood samples were taken at diagnosis, before start of treatment. Healthy control sera were obtained from blood donors at the local blood donation center. Blood samples were collected in 3.5 ml BD SST II Advance serum separator tubes (Becton Dickinson, Franklin Lakes, N.J., USA) and centrifuged at 2000×g at 25° C. for 10 min after 30 minutes clotting. The serum samples were stored at −80° C. in the local Pancreatic Biobank until further use. The clinical information describing the study population is summarized in Table 1.

TABLE 1
Study population demographics

| | | No. of | | |
|---|---|---|---|---|
| | Diagnosis | patients | Age | Male:Female |
| HDMSE | Pancreatic cancer | 9 | 69 (46-77) | 4:5 |
| | Benign pancreatic | 9 | 70 (58-77) | 4:5 |
| | disease | | | |
| | Healthy | 9 | 63 (48-70) | 5:4 |
| | Total | 27 | 67 (46-77) | 13:14 |
| ELISA | Pancreatic cancer | 20 | 68.5 (39-78) | 13:7 |
| | St. I-II | | | |
| | Pancreatic cancer | 15 | 67 (48-77) | 8:7 |
| | St. III-IV | | | |
| | Healthy | 20 | 54 (50-63) | 16:4 |
| | Total | 55 | 63 (39-78) | 37:18 |

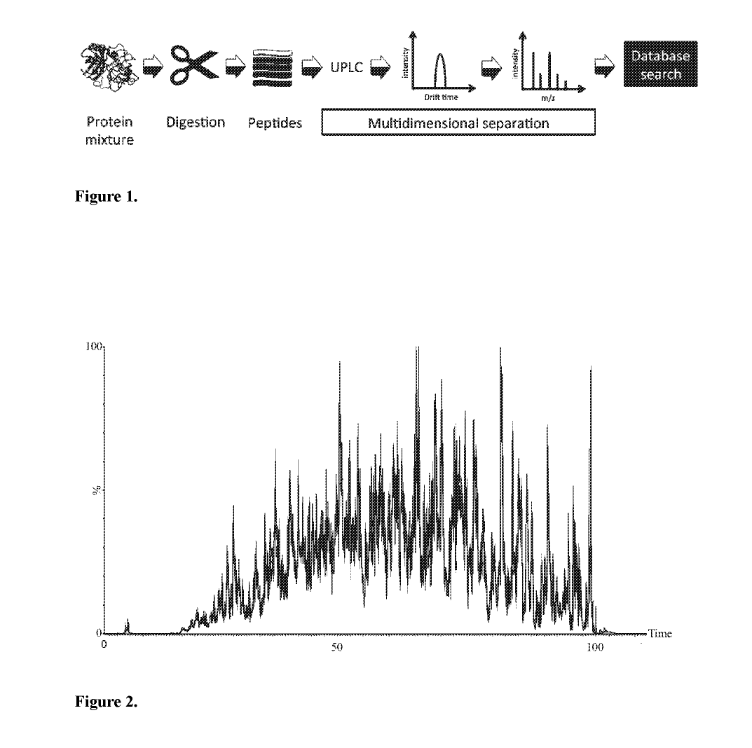

Mass spectrometry and proteomic analysis was performed on a total of 27 serum samples (FIG. 1). The sera were from 9 patients with pancreatic cancer (stages IIA and IIB), 9 patients with benign pancreatic disease and 9 healthy blood donors. Among the benign group, the patients had chronic pancreatitis (n=4), intraductal papillary mucinous neoplasm (IPMN; n=3), serous cystadenoma (n=1) and benign biliary stricture (n=1). Blood samples were collected in BD SST II Advance tubes (serum separator tubes, 3.5 ml, product no. 368498; Becton Dickinson, Franklin Lakes, N.J., USA). The minimum clotting time was 30 min. The samples were centrifuged at 2000×g at 25° C. for 10 min, serum collected and stored in aliquots at −80° C.

To enrich for proteins of low-abundance, each sample was depleted of seven proteins that are highly abundant in serum (albumin, IgG, IgA, transferrin, haptoglobin, antitrypsin, and fibrinogen). Briefly, crude sera (10 μL) were diluted with 180 μL of Buffer A (product no. 5185-5987; Agilent Technologies, Santa Clara, Calif., USA) and then filtered through 0.22 μm spin filter (product no. 5185-5990; Agilent Technologies) by spinning at 1000×g at room temperature for 5 minutes. Diluted serum was injected on a multiple affinity removal system spin cartridge (product no. 5188-6408; Agilent Technologies) in Buffer A. The bound proteins were eluted with Buffer B (product no. 5185-5988; Agilent Technologies).

The proteins were reduced with 10 mM dithiothreitol (Sigma-Aldrich, Si. Louis, Mo., USA) for 1 h at 56° C. and alkylated using 50 mM iodoacetamide (Sigma-Aldrich) for 30 min, kept dark at room temperature. Following this procedure, buffer exchange was performed with 50 mM ammonium bicarbonate buffer (pH 7.6) by using a 10 kDa cut-off spin filter (YM10 filter, AMICON, Millipore, Billerica, Mass., USA). The samples were digested with sequencing grade trypsin (Promega, Madison, Wis., USA) in ratio 1:50 w/w (trypsin: protein) overnight at 37° C. The reaction was stopped by addition of 30 μL of 1% formic acid (Sigma-Aldrich). The resulting protein digests were dried on speed vacuum centrifugation and resuspended with 1% formic acid prior injection. Samples were diluted 1:1 with 10 fmol/μL of yeast alcohol dehydrogenase (ADH) internal standard tryptic digest (Waters, Milford, Mass., USA) before analysis.

Complex tryptic peptide mixtures were separated using nanoscale chromatography performed using a nanoACQUITY UPLC (Waters). One-dimensional reversed phase (RP) nanoACQUITY experiments with trapping were performed.

Mobile phases A and B were 0.1% (v/v) formic acid in water and 0.1% (v/v) formic acid in acetonitrile, respectively. Following desalting of the peptides on a Symmetry C18 5 μm, 2 cm×180 μm trap column (Waters), a reversed phase gradient was employed to separate peptides using 5 to 40% acetonitrile in water over 90 minutes on a 25 cm×75 μm analytical RP column (Waters, USA) at a flow rate of 300 nL/min and a constant temperature of 35° C.

Analysis of the complex peptide mixtures was performed using a SYNAPT G2-Si HDMS mass spectrometer (Waters, Manchester, UK) operated in a data-independent manner coupled with ion mobility (HDMS^{E}) [13]. The mass spectrometer was operated in positive ESI resolution mode with resolution of >250,000 FWHM. In all experiments the mass spectrometer was programmed to step between low energy (4 eV) and elevated (14-40 eV) collision energies on the Triwave collision cell, using a scan time of 0.9 s per function over 50-2000 m/z.

HDMS^{E} data-independent analysis provides detection of all precursor and product ions with accurate mass measurement. Alignment of precursor and product ions by drift and retention time aids peptide identification by assignment of product ions to parent ions during data processing and database searching [14, 15]. Protein identifications and quantification information were obtained by using UniProt human database Progenesis QI for Proteomics version 1.0 and a human UniProt database. Gene ontology annotations were retrieved from the PANTHER classification system [16].

The experiment was normalized using the peptides of the added internal standard protein ADH from yeast. Protein lists were processed using Qlucore Omics Explorer version 3.0. Statistical analysis was performed using log 2-transformed normalized abundances. Multiple group comparison was conducted with the ANOVA test. Hierarchical clustering and principal component analysis (PCA) were employed to visualize any statistically significant differences between the groups. Protein interaction maps were obtained from the Search Tool for the Retrieval of Interacting Genes/Proteins (STRING) database version 9.1 containing known and predicted physical and functional protein-protein interactions [17]. A p-value less than 0.05 was considered statistically significant.

The pancreatic cancer patients included in this study all underwent pancreatic resection with curative intent. All patients were treated with adjuvant chemotherapy after surgery that lasted for 6 months (median 6 cycles).

In the first development phase of the study, single samples from each group were injected in triplicate. The HDMS^{E} platform generates high peak capacity that maximizes the protein identification, whilst retaining label-free quantification capabilities. To assess the analytical reproducibility of the LC/MS acquisition and data processing, we calculated the intensity differences between peaks from triplicate acquisitions of the same serum sample. Some 4801 peptides were identified within the data, for each cycle run.

In the second part of the assay development, we continued analyzing all 27 patient samples by duplicate injections. The HDMS^{E} data files were interrogated with Progenesis QI for Proteomics for protein identification and quantification. The resulting proteins were then subjected to stringent independent validation within the software. The differential protein quantification was performed by calculating the sum of all unique normalized peptide ion abundances for a specific protein on each run and then comparing mean values between samples. As the study was conducted over a substantial time period, a normalization procedure was important, utilizing ADH, as an internal control in all clinical samples (for details see Experimental). We also performed the study by having the QC run as the calibrant within the assay, at frequency as the 8th sample within the analysis cycle.

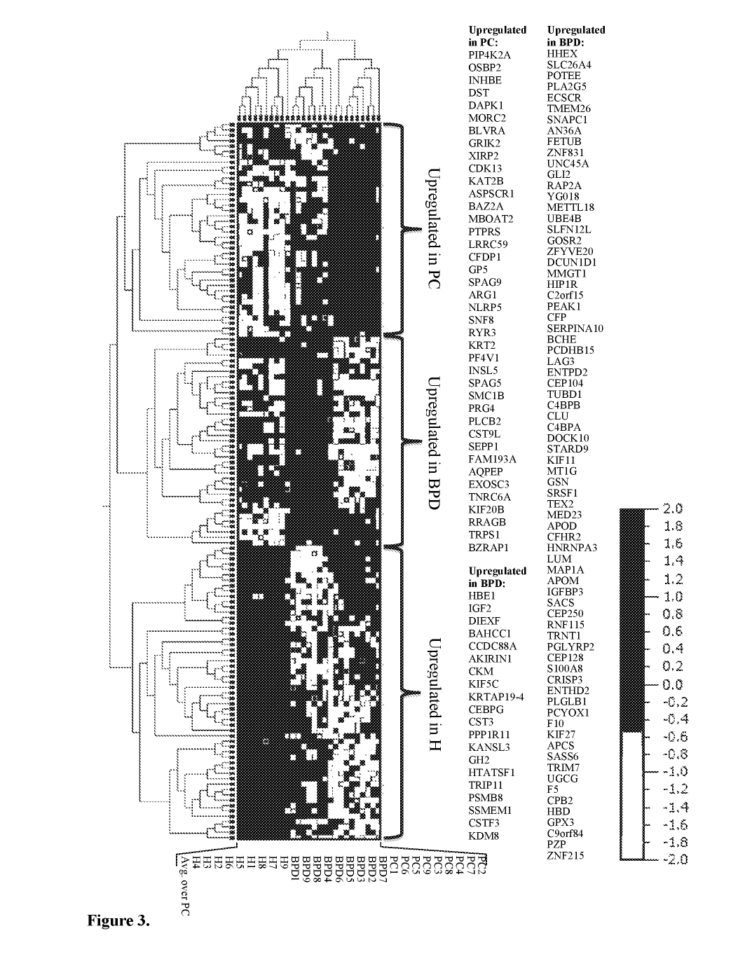

To define if protein expression profiles were distinct between pancreatic cancer and control samples, we performed unsupervised hierarchical clustering on log-transformed baseline protein concentrations, as outlined in FIG. 3. A two-way clustering approach was applied in order to allow a meaningful clustering of both proteins and samples.

Listed sequences of proteins and polypeptides by use of the standard one letter codes representing the constituting amino acids. The order of the amino acids written from left to right correspond to the sequence of the respective protein or polypeptide from the amino- to the carboxylic acid ending thereof. The sequence of endogenous proteins or polypeptides are assigned a code of the format SeqIDNon, wherein “n” is an integer number, which code the endogenous protein or polypeptide may be referred to herein as an alternative to the corresponding gene or commonly accepted name, as listed in table 7. The sequence of a typical fragment or typical fragments, which may be produced in-vitro by employment of trypsin to fully or partly digest the original endogenous protein or polypeptide by cleavage at the carboxylic acid side of lysine (K) and arginine (R) residues as described herein, is/are analogously herein alternatively referred to a as a code of format SeqIDNon, wherein “n” is an integer number, wherein table 7 lists which endogenous protein or polypeptide the fragment originates from.

ELISA was used for quantitative analysis on a total of 55 serum samples, from the patient group described in table 1. Biomarkers used for ELISA analysis were from the group consisting of GP5, HNRNPC, G7d, KAT2B, KIF20B, SMC1B and SPAG5. Serum samples were measured using enzyme-linked immunosorbent assay (ELISA) kits (Cloud-Clone Corp., Huston, Tex., USA) for GP5 according to the manufacturer's instructions. Briefly, 100 μl serum samples, quality control or standards were added to microtiter plates pre-coated with rabbit polyclonal antibody raised against recombinant biomarker and incubated for 2 h at 37° C. After the content of the wells was removed, the wells were further incubated with biotine-conjugated detection antibody for 1 h at 37° C. The wells were then washed and incubated with the detection reagent, avidin conjugated to Horse radish peroxidase (HRP) for 30 min at 37° C. before adding the TMB substrate to exhibit a change of color in wells containing biomarker, biotin conjugated antibody and the enzyme conjugated avidin. The enzymatic reaction was terminated by adding sulphuric acid solution and the color change was measured spectrophotometrically at a wavelength of 450 nm on Labsystems Multiscan Plus plate reader. The concentration of biomarker in the samples was calculated from optical density (O.D.) values using DeltaSoft JV software (BioMetallics Inc., Princeton, N.J., USA). The recombinant biomarker sequences used for antibody production comprised two of three peptides applied for identification and quantification of the biomarkers with HDMS^{E}.

CA19-9 levels were analyzed at the department of clinical chemistry, Skåne University Hospital, Lund, Sweden, according to standardized method. In short, Single-stage immunometric sandwich method ElectroChemiLuminiscence-Immunoassay (ECLI) detection technique based on Reuthenium (Ru) derivatives was used. Samples (antigen-Ag), mouse monoclonal anti-CA19-9 antibodies conjugated with biotin (conjugate, biotin-MAk1) and mouse monoclonal anti-CA19-9-antibodies labeled with Ru (Pak2-Ru) forms a sandwich complex (Biotin-MAk1---Ag---Pak2-Ru). Paramagnetic particles covered with streptavidin are added. The sandwich complex binds to paramagnetic particles (solid phase) through Biotin-Streptavidin-interaction thus forming a Streptavidin---Biotin-MAk1---Ag---Pak2-Ru-formation. The antigen-antibody complex is detected by an electrochemical reaction which results in the emission of light (electrochemiluminescence), the intensity of which is measured. The light intensity is directly proportional to the CA19-9 concentration in the sample.

Furthermore, the pancreatic cancer patients included in this study all underwent pancreatic resection with curative intent. Tumor sections of 4 μm on object glass were deparaffinized in xylene and rehydrated in graded ethanol.

The R statistical programming language was used for all statistical analysis. Receiver operating characteristic (ROC) curves were drawn to visualize the interrelationship between sensitivity and specificity. The area under the curves (AUC) were calculated and sensitivities at defined specificities were calculated to test for the performance of the biomarkers for differential diagnosis of cancer. P-values ≤0.05 were considered as statistically significant.

The results of these assays were analyzed using an optimal clustering algorithm. After measurement assays results, single and multivariate analysis methods were conducted. Fisher's linear discriminant analysis (LDA) was used to determine the weighted sum of the variables that provides the optimal discrimination between two diagnoses (such as Cancer vs Healthy). For each sample, the following formula was used:

W=(C̅−H̅)S⁻¹{x−½(C̅+H̅)}

where x is the sample's OD (optical density) value, C is the mean of the samples with a Cancer diagnosis, H is the mean of the samples with a Healthy diagnosis and S is the covariance matrix.

Statistical analysis was performed for proteins GP5, HNRNPC, G7d, KAT2B, KIF20B, SMC1B and SPAG5 and biomarker CA19.9. Also combinations of GP5+CA19.9, GP5+HNRNPC, GP5+HNRNPC+CA19.9, GP5+HNRNPC+KIF20B and GP5+SPAG5+KIF20B were evaluated. Optimal cut-offs were calculated by the LDA method: Cut-off=½(Cbar+Hbar), which corresponds to 0 on the boxplots.

Results

As a measure of analytical reproducibility of the LC/MS acquisition and data processing, intensity differences between peaks from triplicate acquisitions of the same serum sample were calculated. Some 4801 peptides were identified within the data, for each cycle run. All triplicate data points showed less than 4% variation in intensity, while the chromatographic reproducibility was found to have 2-4% RSD. These shotgun analysis data are illustrated in FIG. 2, with triplicate LC-MS overlayed BPI chromatograms where the platform performance can be viewed to be highly constant over the entire cycle run, going from hydrophilic to hydrophobic peptide sequences. The MS data from the different replicates were clustered tightly and showed that there is a high reproducibility.

All 27 patient samples were analyzed using duplicate injections. Within this part of the study, we generated a data output of 71,209 distinct features. The HDMS^{E} data files were interrogated with Progenesis QI for Proteomics for protein identification and quantification. The resulting proteins were then subjected to stringent independent validation within the software. By using an identification criterion of 80% peptide probability and 99% protein probability, a total number of 7,947 unique peptides and 715 unique proteins were identified using a false discovery rate <0.5%.

The pancreatic cancer patients included in the HDMS^{E} study all underwent pancreatic resection with curative intent. Pathologically, the tumors were located in the pancreatic head, with a median size of 3.0 cm (0.3-4.0 cm). All patients were diagnosed with T3 tumors, referring to that the tumor did not involve the surrounding major vessels of the pancreas. Out of these T3 patients, 7 patients were diagnosed with N1 stage, i.e., lymph node metastases, while 2 of the patients had NO stage. This means that there were no lymph node metastases diagnosed. Lymphovascular invasion was detected in 5 out of the 9 patients. The patients were further characterized by having perineural invasion (neural infiltration) in 7 out of the 9 patients. In addition, we found that 7 out of 9 patients had moderately differentiated tumors while 2 patients had poorly differentiated tumors.

All patients were treated with adjuvant chemotherapy after surgery that lasted for 6 months (median 6 cycles). With a median follow-up after 386 days (258-658 days), we could clinically verify that all patients were alive. A summary of all the clinical and histopathological data and characteristics that we built within the biobank administration database are listed in Tables 2 and 5.

TABLE 2
Clinical and histopathological characteristics of the pancreatic cancer patients

| | | | Tumor | | | | | | Adjuvant | | Disease |
|---|---|---|---|---|---|---|---|---|---|---|---|
| Age | Sex | Stage | size | pT | pN | LVI | PNI | Grade | chemotherapy | Follow-up | status |
| 77 | F | IIB | 2.8 cm | pT 3 | pN1 (4/26) | 1 | 1 | 2 | GEM; 6 cycles | 658 days | Alive |
| 70 | M | IIA | 0.3 cm | pT 3 | pN0 (0/38) | 0 | 0 | 3 | 5-FU; 10 cycles | 581 days | Alive |
| 67 | M | IIB | 3.0 cm | pT 3 | pN1 (9/21) | 0 | 1 | 2 | GEM; 6 cycles | 589 days | Alive |
| 69 | M | IIA | 4.0 cm | pT 3 | pN0 (0/27) | 0 | 1 | 3 | GEM, CAP; 5 cycles | 455 days | Alive |
| 69 | F | IIB | 3.2 cm | pT 3 | pN1 (11/28) | 1 | 1 | 2 | GEM; 6 cycles | 386 days | Alive |
| 62 | F | IIB | 1.5 cm | pT 3 | pN1 (16/45) | 1 | 1 | 2 | GEM; 6 cycles | 331 days | Alive |
| 70 | F | IIB | 3.8 cm | pT 3 | pN1 (4/25) | 1 | 1 | 2 | GEM; 6 cycles | 351 days | Alive |
| 63 | M | IIB | 4.0 cm | pT 3 | pN1 (11/13) | 1 | 1 | 2 | GEM; 6 cycles | 308 days | Alive |
| 46 | F | IIB | 2.9 cm | pT 3 | pN1 (6/17) | 0 | 0 | 2 | GEM; 6 cycles | 258 days | Alive |

5-FU, 5-fluorouracil; CAP, capecitabine; GEM, gemcitabine; LVI, lymphovascular invasion; PNI, perineural invasion.

Gene ontology analysis was undertaken to assess the holistic biological role and molecular function of the identified proteins. The annotation highlighted a significant portion of species involved in both binding and catalytic processes. In terms of biological process the proteins were represented most highly by those involved in metabolic and cellular processes (see FIG. 5). This is in line what the pancreas study team was expecting. Similar ontology groupings were identified by other research groups in recent studies [18, 19].

As can be seen in FIG. 3, we were able to find group specific regulation in each study group in the resulting heat-map for 134 differentially expressed proteins (p<0.0009). Further, the analysis showed several clusters that could be used for classification purposes. In particular, one cluster containing 40 proteins showed a significant up-regulation in the pancreatic cancer group as shown in Table 3. By these statistical calculations, low q-values (all below 0.005), were provided indicating a low false discovery rate.

TABLE 3
Up-regulated proteins in pancreatic cancer according to two-way unsupervised hierarchical clustering

| Gene | | | | | |
|---|---|---|---|---|---|
| names | Accession | Description | Function | p-value | q-value |
| PIP4K2A | P48426 | Phosphatidylinositol-5- | 1-phosphatidylinositol- | 0.000440686 | 0.002864456 |
| | | phosphate 4-kinase | 4-phosphate 5-kinase | | |
| | | type-2 alpha | activity; ATP binding | | |
| OSBP2 | Q969R2 | Oxysterol-binding | Lipid transport | 0.000393512 | 0.002650246 |
| | | protein 2 | | | |
| INHBE | P58166 | Inhibin beta E chain | Growth | 0.000792528 | 0.004265124 |
| DST | O94833 | Bullous pemphigoid | Actin cytoskeleton; | 0.000195048 | 0.001621617 |
| | | antigen 1, isoforms | axogenesis; cell cycle | | |
| | | 6/9/10 | arrest; cell motility | | |
| DAPK1 | P53355 | Death-associated | Apoptotic process; | 0.000165779 | 0.001428092 |
| | | protein kinase 1 | regulation of | | |
| | | | autophagy; ATP | | |
| | | | binding | | |
| MORC2 | Q9Y6X9 | MORC family CW-type | ATP binding | 0.000393969 | 0.002650246 |
| | | zinc finger protein 2 | | | |
| BLVRA | P53004; | Biliverdin reductase A | Biliverdin reductase | 0.000275787 | 0.00207566 |
| | Q6IPR1 | | activity; oxidation- | | |
| | | | reduction process | | |
| GRIK2 | Q13002 | Glutamate receptor, | Glutamate receptor | 0.000388802 | 0.002650246 |
| | | ionotropic kainate 2 | signaling pathway | | |
| XIRP2 | A4UGR9 | Xin actin-binding | Actin cytoskeleton | 0.000254006 | 0.001952844 |
| | | repeat-containing | organization | | |
| | | protein 2 | | | |
| CDK13 | Q14004 | Cell division protein | Cyclin K-CDK13 | 2.02E−05 | 0.00039023 |
| | | kinase 13 | complex; ATP binding | | |
| KAT2B | Q92831 | Histone | Histone | 2.18E−06 | 0.000103756 |
| | | acetyltransferase | acetyltransferase | | |
| | | KAT2B | activity; cell cycle | | |
| | | | arrest | | |
| ASPSCR1 | Q9BZE9 | Tether containing UBX | Glucose homeostasis | 5.10E−06 | 0.000158818 |
| | | domain for GLUT4 | | | |
| BAZ2A | Q9UIF9 | Bromodomain adjacent | Chromatin silencing | 0.000143598 | 0.001341717 |
| | | to zinc finger domain | complex; histone | | |
| | | protein 2A | deacetylation | | |
| MBOAT2 | Q6ZWT7 | Lysophospholipid | 1-acylglycerol-3- | 0.000746082 | 0.004167566 |
| | | acyltransferase 2 | phosphate O- | | |
| | | | acyltransferase activity; | | |
| | | | lipid metabolism; | | |
| | | | phospholipid | | |
| | | | metabolism | | |
| PTPRS | Q13332 | Receptor-type tyrosine- | Cell adhesion; | 9.70E−05 | 0.000996004 |
| | | protein phosphatase S | transmembrane receptor | | |
| | | | protein tyrosine | | |
| | | | phosphatase activity | | |
| LRRC59 | Q96AG4 | Leucine-rich repeat- | Required for nuclear | 0.000220637 | 0.00173358 |
| | | containing protein 59 | import of FGF1 | | |
| CFDP1 | Q9UEE9 | Craniofacial | Cell adhesion; negative | 0.000363602 | 0.002574013 |
| | | development protein 1 | regulation of fibroblast | | |
| | | | apoptotic process; | | |
| | | | regulation of cell | | |
| | | | proliferation | | |
| GP5 | P40197 | Platelet glycoprotein V | Blood coagulation; cell | 0.000230272 | 0.001789617 |
| | | | adhesion; cell-matrix | | |
| | | | adhesion | | |
| SPAG9 | O60271 | C-Jun-amino-terminal | Activation of JUN | 2.69E−05 | 0.000436741 |
| | | kinase-interacting | kinase activity | | |
| | | protein 4 | | | |
| ARG1 | P05089 | Arginase-1 | Arginase activity; | 7.98E−05 | 0.000897303 |
| | | | cellular response to | | |
| | | | transforming growth | | |
| | | | factor beta stimulus | | |
| NLRP5 | P59047 | NACHT, LRR and PYD | ATP binding; neuron | 8.13E−05 | 0.000897303 |
| | | domains-containing | death; regulation of | | |
| | | protein 5 | RNA stability; | | |
| | | | regulation of protein | | |
| | | | stability | | |
| SNF8 | Q96H20 | Vacuolar-sorting | Endosomal transport; | 3.19E−05 | 0.000475532 |
| | | protein SNF8 | regulation of | | |
| | | | transcription from RNA | | |
| | | | polymerase II promoter | | |
| RYR3 | Q15413 | Ryanodine receptor 3 | Calcium ion binding; | 4.38E−05 | 0.000590852 |
| | | | cellular response to | | |
| | | | ATP | | |
| KRT2 | P35908 | Keratin, type II | Keratinization | 8.16E−05 | 0.000897303 |
| | | cytoskeletal 2 | | | |
| | | epidermal | | | |
| PF4V1 | P10720 | Platelet factor 4 variant | Cell chemotaxis; | 4.53E−06 | 0.000158818 |
| | | | immune response | | |
| INSL5 | Q9Y5Q6 | Insulin-like peptide | Member of the insulin | 0.000151395 | 0.001361213 |
| | | INSL5 | superfamily | | |
| SPAG5 | Q96R06 | Astrin | Activation of JUN | 4.47E−05 | 0.000590852 |
| | | | kinase activity | | |
| SMC1B | Q8NDV3 | Structural maintenance | DNA repair; ATP | 4.43E−05 | 0.000590852 |
| | | of chromosomes protein 1B | binding | | |
| PRG4 | Q92954 | Proteoglycan 4 | Cell proliferation; | 3.02E−05 | 0.000468868 |
| | | | immune response | | |
| PLCB2 | Q00722 | 1-phosphatidylinositol- | Activation of | 8.21E−06 | 0.000202444 |
| | | 4,5-bisphosphate | phospholipase C | | |
| | | phosphodiesterase beta-2 | activity; signal | | |
| | | | transducer activity | | |
| CST9L | Q9H4G1 | Cystatin-9-like | Cysteine-type | 1.49E−05 | 0.000344803 |
| | | | endopeptidase inhibitor | | |
| | | | activity | | |
| SEPP1 | P49908 | Selenoprotein P | Selenium binding; | 1.07E−06 | 6.94E−05 |
| | | | response to oxidative | | |
| | | | stress | | |
| FAM193A | P78312 | Protein FAM193A | Unknown | 4.16E−06 | 0.000158818 |
| AQPEP | Q6Q4G3 | Aminopeptidase Q | Metallopeptidase | 5.51E−06 | 0.000161882 |
| | | | activity | | |
| EXOSC3 | Q9NQT5 | Exosome complex | 3′-5′-exoribonuclease | 0.000154207 | 0.001361213 |
| | | exonuclease RRP40 | activity; RNA | | |
| | | | metabolic process | | |
| TNRC6A | Q8NDV7 | Trinucleotide repeat- | Fc-epsilon receptor | 3.17E−05 | 0.000475532 |
| | | containing gene 6A | signaling pathway; | | |
| | | protein | cellular response to | | |
| | | | starvation | | |
| KIF20B | Q96Q89 | Kinesin-like protein | ATP binding; cell cycle | 7.89E−05 | 0.000897303 |
| | | KIF20B | arrest | | |
| RRAGB | Q5VZM2; | Ras-related GTP- | GTP binding; positive | 0.000197655 | 0.001624408 |
| | Q7L523 | binding protein B | regulation of TOR | | |
| | | | signaling | | |
| TRPS1 | Q9UHF7 | Zinc finger | Transcriptional | 2.99E−05 | 0.000468868 |
| | | transcription factor | represser of GATA- | | |
| | | Trps1 | regulated genes | | |
| BZRAP1 | O95153 | Peripheral-type | Benzodiazepine | 0.000659306 | 0.003832553 |
| | | benzodiazepine | receptor binding | | |
| | | receptor-associated | | | |
| | | protein 1 | | | |

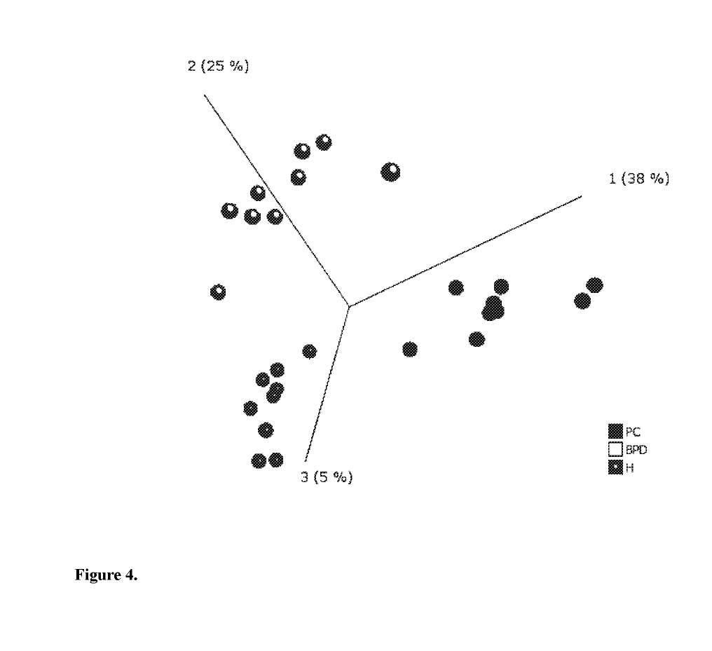

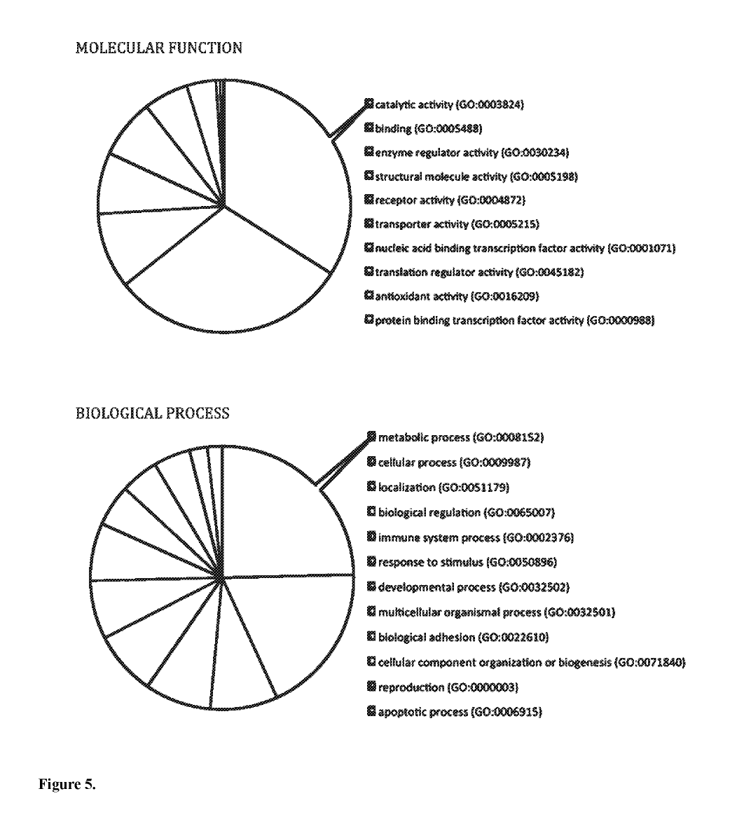

These distinct protein profile signatures observed between pancreatic cancer and control phenotypes after clustering analyses were further confirmed by PCA. In the PCA score plot (FIG. 4), samples that have similar protein expression profiles fall close to each other. This was found to correlate well with the clinical stratification. We also observed a larger variation in the protein expressions among the pancreatic cancer and benign cases compared with the healthy samples. This is illustrated in the PCA plot by the more scattered distribution of cancer samples (blue) and benign cases (yellow) compared with healthy samples (pink). These findings suggest that the cancer and benign population are more heterogeneous than the corresponding healthy population. Furthermore, as can be seen in the plot, the first principal component contains 38% of the total variance and clearly sets the pancreatic cancer group apart from the rest of the subtypes. Overall, these data provide evidence that the pancreatic cancer cohort can be stratified by our unique group of proteins.

Using ELISA assay, it was found that out of GP5, HNRNPC, G7d, KAT2B, KIF20B, SMC1B and SPAG5 proteins, GP5 (Human platelet glycoprotein V) provided excellent sensitivity at a high level of specificity, as summarized in Table 4.

TABLE 4
ELISA Biomarker trials

| | Sensitivity | at specificity | AUC of ROC |
|---|---|---|---|
| Biomarker | (%) | (%) | (%) |
| GP5 | 88 | 80 | 86.67 |
| HNRNPC | 66.67 | 80 | 58.89 |
| SMC1B | 44.44 | 100 | 61.11 |
| G7d | 44.44 | 90 | 58.89 |
| KAT2B | 77.7 | 50 | 53.89 |
| KIF20B | 44.44 | 100 | 60 |
| SPAG5 | 44.44 | 90 | 54.44 |
| CA.19.9 | 88.89 | 90 | 85.56 |
| GP5 + CA.19.9 | 88.89 | 90 | 90 |
| GP5 + HNRNPC | 100 | 80 | 94.44 |
| GP5 + HNRNPC + CA.19.9 | 100 | 100 | 100 |
| GP5 + HNRNPC + KIF20B | 100 | 90 | 96.67 |
| GP5 + SPAG5 + KIF20B | 100 | 90 | 94.44 |

A full summary for GP5 abundance for all patients of the study using ELISA is summarized in Table 5.

TABLE 5
GPS Study population demographics

| Pancreatic cancer, | | | Pancreatic cancer, | | | Healthy | | |
|---|---|---|---|---|---|---|---|---|
| stages I-II | | | stages III-IV | | | control | | |
| GP5 | | Gen- | GP5 | | Gen- | GP5 | | Gen- |
| (μg/L) | Age | der | (μg/L) | Age | der | (μg/L) | Age | der |
| 1.741 | 63 | F | 38.152 | 75 | F | 2.522 | 59 | M |
| 5.401 | 60 | F | 3.832 | 69 | F | 1.173 | 52 | M |
| 3.805 | 39 | F | 2.674 | 59 | M | 0.955 | 55 | M |
| 1.997 | 75 | M | 4.245 | 62 | F | 1.828 | 50 | M |
| 2.318 | 71 | M | 3.496 | 69 | M | 0.687 | 51 | M |
| 1.647 | 69 | M | 4.571 | 61 | F | 1.038 | 54 | F |
| 1.719 | 77 | M | 2.538 | 68 | M | 1.132 | 54 | F |
| 2.98 | 70 | M | 4.87 | 67 | M | 1.072 | 62 | M |
| 1.554 | 64 | M | 2.797 | 48 | F | 2.654 | 53 | F |
| 1.211 | 63 | M | 2.563 | 76 | M | 1.644 | 60 | M |
| 2.399 | 78 | M | 10.03 | 72 | F | 0.646 | 63 | M |
| 1.237 | 68 | F | 6.052 | 58 | M | 1.409 | 53 | M |
| 1.609 | 66 | F | 4.721 | 66 | F | 1.638 | 58 | M |
| 2.242 | 75 | F | 5.037 | 77 | M | 1.529 | 51 | F |
| 3.231 | 73 | M | 2.726 | 66 | M | 0.435 | 62 | M |
| 4.078 | 75 | M | | | | 0.816 | 52 | M |
| 3.437 | 69 | M | | | | 1.641 | 54 | M |
| 3.968 | 68 | M | | | | 1.929 | 53 | M |
| 1.835 | 65 | F | | | | 1.197 | 62 | M |
| 2.727 | 64 | M | | | | 1.396 | 62 | M |
| 2.5568 | 68.5 | — | 6.5536 | 67 | — | 1.36705 | 54 | — |

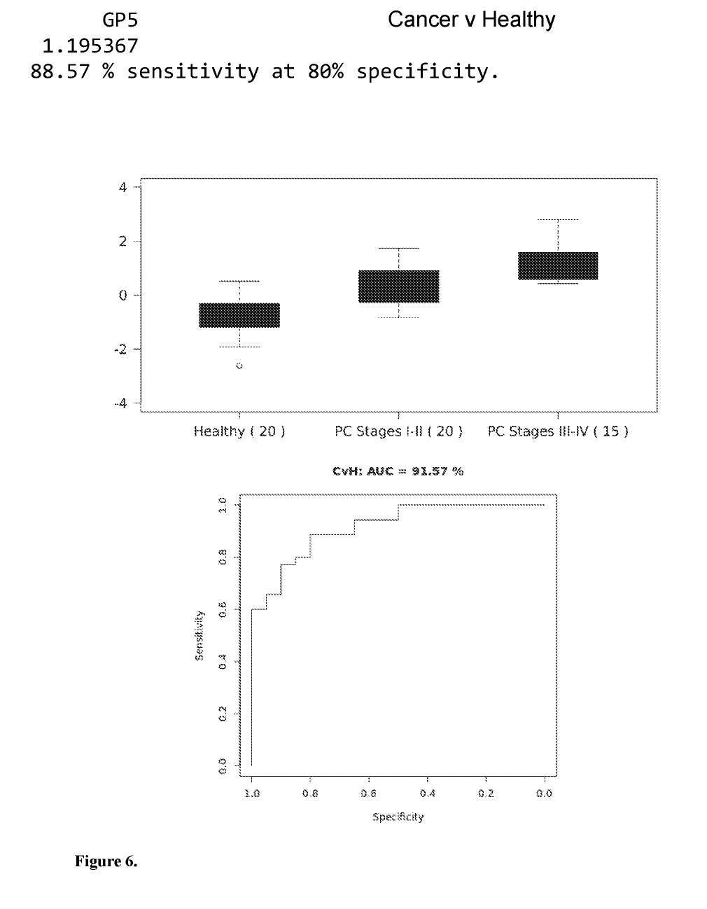

FIG. 6 shows in detail that GP5 provides both high sensitivity and specificity for determining a subject's probability to suffer from pancreatic cancer. The AUC for the discrimination of pancreatic cancer from healthy controls reached 91%; sensitivity 77% at 90% specificity.

The optimal cut-off for GP5 for pancreatic cancer prediction was calculated using the linear discriminant (LDA) formula to log(GP5)≤0.934, that is a GP5 abundance of ≤1.978 μg/L for a healthy individual.

FIG. 7 shows GP5 used together with CA19.9 for pancreatic cancer prediction, reaching an AUC for the discrimination of pancreatic cancer from healthy controls reached 96%; sensitivity 97% at 90% specificity.

Table 6 shows the results from ELISA trials of measuring a combination of GP5 and other biomarkers. Here GP5 abundance together with HNRNPC and CA19.9 provides an AUC of 95%, which illustrates an excellent predictability of pancreatic cancer for the patient group.

TABLE 6
Combining GP5 with other biomarkers

| | Sensitivity | at specificity | AUC of ROC |
|---|---|---|---|
| Biomarker | (%) | (%) | (%) |
| GP5 | 90.00 | 81.82 | 90 |
| HNRNPC | 40.00 | 90.91 | 55 |
| CA.19.9 | 95.00 | 90.91 | 92.73 |
| GP5 + HNRNPC | 90.00 | 90.91 | 92.73 |
| GP5 + CA.19.9 | 95.00 | 90.91 | 94.09 |
| HNRNPC + CA.19.9 | 95.00 | 90.91 | 93.64 |
| GP5 + HNRNPC + CA.19.9 | 90.00 | 100 | 95.00 |

FIG. 8 shows GP5 used for differentiating between pancreatic cancer stages I and II vs. stages III and IV. The AUC for the discrimination of pancreatic cancer Stages I-II from Stages III-IV reached 83%; sensitivity 66.6% at 90% specificity.

Protein and Polypeptide Sequences

Below follows a table in which above listed codes of endogenous proteins or polypeptides are related to the corresponding gene or commonly accepted names or further description.

TABLE 7
List of proteins or polypeptides

| Assigned | | | |
|---|---|---|---|
| Code | Gene name | Commonly accepted name or description | Fragment codes |
| SeqIDNo117 | HBE1 | Hemoglobin subunit epsilon | SeqIDNo1- |
| | | | SeqIDNo2 |
| SeqIDNo118 | KIF20B | Kinesin-like protein KIF20B | SeqIDNo3- |
| | | | SeqIDNo10 |
| SeqIDNo119 | ZNF831 | Zinc finger protein 831 | SeqIDNo11- |
| | | | SeqIDNo14 |
| SeqIDNo120 | SPAG5 | Sperm-associated antigen 5 | SeqIDNo14- |
| | | | SeqIDNo18 |
| SeqIDNo121 | PLGLB1 | Plasminogen-related protein B | SeqIDNo19- |
| | | | SeqIDNo26 |
| SeqIDNo122 | FAM193A | Protein FAM193A | SeqIDNo27- |
| | | | SeqIDNo28 |
| SeqIDNo123 | UBXN2A | UBX domain-containing protein 2A | SeqIDNo29 |
| SeqIDNo124 | GP5 | Platelet glycoprotein V | SeqIDNo30- |
| | | | SeqIDNo32 |
| SeqIDNo125 | AN36A | Ankyrin repeat domain-containing protein 36 | SeqIDNo33- |
| | | | SeqIDNo40 |
| SeqIDNo126 | SMC1B | Structural maintenance of chromosomes | SeqIDNo41- |
| | | protein 1B | SeqIDNo49 |
| SeqIDNo127 | TOPAZ1 | Uncharacterized protein C3orf77 | SeqIDNo50- |
| | | | SeqIDNo68 |
| SeqIDNo128 | BOD1L1 | Biorientation of chromosomes in cell division | SeqIDNo69- |
| | | protein 1-like | SeqIDNo81 |
| SeqIDNo129 | CASP16 | Putative caspase-14-like protein | SeqIDNo82 |
| SeqIDNo130 | KRTAP19-4 | Keratin-associated protein 19-4 | SeqIDNo83 |
| SeqIDNo131 | DNAJC9-AS1 | Putative uncharacterized protein C10orf103 | SeqIDNo84 |
| SeqIDNo132 | HNRNPCL1 | Heterogeneous nuclear ribonucleoprotein C- | SeqIDNo85- |
| | | like 1 | SeqIDNo86 |
| SeqIDNo133 | SSMEM1 | Uncharacterized protein C7orf45 | SeqIDNo87 |
| SeqIDNo134 | LINC00052 | Putative transmembrane protein 83 | SeqIDNo88- |
| | | | SeqIDNo89 |
| SeqIDNo135 | SAPCD1 | Protein G7d | SeqIDNo90 |
| SeqIDNo136 | OR10J5 | Olfactory receptor 10J5 | SeqIDNo91- |
| | | | SeqIDNo94 |
| SeqIDNo137 | PAIP2B | Polyadenylate-binding protein-interacting | SeqIDNo95- |
| | | protein 2B | SeqIDNo96 |
| SeqIDNo138 | LINC00587 | Putative uncharacterized protein C9orf107 | SeqIDNo97 |
| SeqIDNo139 | KRTAP19-5 | Keratin-associated protein 19-5 | SeqIDNo98 |
| SeqIDNo140 | UBE2U | Ubiquitin-conjugating enzyme E2 U | SeqIDNo99- |
| | | | SeqIDNo101 |
| SeqIDNo141 | CXorf28 | Putative uncharacterized protein CXorf28 | SeqIDNo102 |
| SeqIDNo142 | CDRT15 | CMT1A duplicated region transcript 15 | SeqIDNo103 |
| | | protein | |
| SeqIDNo143 | COMTD1 | Catechol O-methyltransferase domain- | SeqIDNo104 |
| | | containing protein 1 | |
| SeqIDNo144 | GLIPR1L2 | GLIPR1-like protein 2 | SeqIDNo105 |
| SeqIDNo145 | PRRC2C | Protein BAT2-like 2 | SeqIDNo106- |
| | | | SeqIDNo111 |
| SeqIDNo146 | KV103 | Ig kappa chain V-I region Bi | SeqIDNo112- |
| | | | SeqIDNo113 |
| SeqIDNo147 | KRTAP13-2 | Keratin-associated protein 13-2 | SeqIDNo114 |
| SeqIDNo148 | CLLU1OS | Putative chronic lymphocytic leukemia up- | SeqIDNo115- |
| | | regulated protein 1 opposite strand transcript | SeqIDNo116 |
| | | protein | |

### Examples

When carrying out a method of the invention, a subject's probability to suffer from pancreatic cancer relative a reference subject is obtained. Below follows examples of various scenarios according to different embodiments. The skilled person will readily understand how to carry out the invention and interpret the results in an optimal way.

Example 1—The subject is a person not diagnosed with pancreatic cancer and the reference subject is a healthy individual which is known, to a high degree of certainty, to not suffer from pancreatic cancer.

When carrying out a method of the invention, the outcome may be one of the following two likely outcomes: A—the probability of the subject to suffer from pancreatic cancer is found to be significantly higher than the probability of the reference subject to suffer from pancreatic cancer. B—no significant difference between the subject's and the reference subject's probability to suffer from pancreatic cancer can be detected. In the case of outcome A, a further investigation of the subject, or other appropriate measures like e.g. frequent monitoring of other signs of pancreatic cancer, may be warranted as the subject may be suspected to suffer from pancreatic cancer. In the case of outcome B, the results may be interpreted as negative, i.e., that no signs of the presence of pancreatic cancer of the subject can be found.

Example 2—The subject is a person diagnosed with pancreatic cancer and the reference subject is the same person but from whom a sample representative of the person's proteome has been collected at a different time, e.g. a different week or a different month.

When carrying out a method of the invention, the outcome may be one of the following three likely outcomes: A—the probability of the subject to suffer from pancreatic cancer is found to be significantly higher than the probability of the reference subject to suffer from pancreatic cancer. B—no significant difference between the subject's and the reference subject's probability to suffer from pancreatic cancer can be detected. C—the probability of the subject to suffer from pancreatic cancer is found to be significantly lower than the probability of the reference subject to suffer from pancreatic cancer. In the case of outcome A, the interpretation may be that the pancreatic cancer has progressed to a more severe state over time, provided that the sample from the subject was collected at a time after the collection of the sample of the reference subject. A change of treatment may thus be motivated. In the case of outcome B, the interpretation may be that the state of the pancreatic cancer has not changed over time. In the case of outcome C, the interpretation may be that the pancreatic cancer has resided to a less severe state over time, provided that the sample from the subject was collected at a time after the collection of the sample of the reference subject.

In the claims, the term “comprises/comprising” does not exclude the presence of other elements or steps. Furthermore, although individually listed, a plurality of means, elements or method steps may be implemented. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second” etc do not preclude a plurality.

### REFERENCES

1. Yachida S, Jones S, Bozic I, Antal T, Leary R, Fu B, et al. Distant metastasis occurs late during the genetic evolution of pancreatic cancer. Nature 2010; 467:1114-7.
2. Decker G A, Batheja M J, Collins J M, Silva A C, Mekeel K L, Moss A A, et al. Risk factors for pancreatic adenocarcinoma and prospects for screening. Gastroenterol Hepatol (N Y) 2010; 6:246-54.
3. Vincent A, Herman J, Schulick R, Hruban R H, Goggins M. Pancreatic cancer. Lancet 2011; 378:607-20.
4. Schnelldorfer T, Ware A L, San M G, Smyrk T C, Zhang L, Qin R, et al. Long-term survival after pancreatoduodenectomy for pancreatic adenocarcinoma: is cure possible? Ann Surg 2008; 247:456-62.
5. Goonetilleke K S, Siriwardena A K. Systematic review of carbohydrate antigen (CA 19-9) as a biochemical marker in the diagnosis of pancreatic cancer. Eur J Surg Oncol 2007; 33:266-70.
6. Tessitore A, Gaggiano A, Cicciarelli G, Verzella D, Capece D, Fischietti M, et al. Serum biomarkers identification by mass spectrometry in high-mortality tumors. Int J Proteomics 2013; 2013:125858.
7. Langley S R, Dwyer J, Drozdov I, Yin X, Mayr M. Proteomics: from single molecules to biological pathways. Cardiovasc Res 2013; 97:612-22.
8. Domon B, Aebersold R. Options and considerations when selecting a quantitative proteomics strategy. Nat Biotechnol 2010; 28:710-21.
9. Kim M S, Pinto S M, Getnet D, Nirujogi R S, Manda S S, Chaerkady R, et al. A draft map of the human proteome. Nature 2014; 509:575-81.
10. Wilhelm M, Schlegl J, Hahne H, Moghaddas Gholami A, Lieberenz M, Savitski M M, et al. Mass-spectrometry-based draft of the human proteome. Nature 2014; 509:582-7.
11. Ansari D, Aronsson L, Sasor A, Welinder C, Rezeli M, Marko-Varga G, et al. The role of quantitative mass spectrometry in the discovery of pancreatic cancer biomarkers for translational science. J Transl Med 2014; 12:87.
12. Bond N J, Shliaha P V, Lilley K S, Gatto L. Improving qualitative and quantitative performance for MS(E)-based label-free proteomics. J Proteome Res 2013; 12:2340-53.
13. Rodriguez-Suarez E, Hughes C, Gethings L, Giles K, Wildgoose J, Stapels M, et al. An ion mobility assisted data independent LC-MS strategy for the analysis of complex biological samples. Curr Anal Chem 2013; 9:199-211.
14. Silva J C, Gorenstein M V, Li G Z, Vissers J P, Geromanos S J. Absolute quantification of proteins by LCMSE: a virtue of parallel MS acquisition. Mol Cell Proteomics 2006; 5:144-56.
15. Silva J C, Denny R, Dorschel C A, Gorenstein M, Kass I J, Li G Z, et al. Quantitative proteomic analysis by accurate mass retention time pairs. Anal Chem 2005; 77:2187-200.
16. Mi H, Muruganujan A, Casagrande J T, Thomas P D. Large-scale gene function analysis with the PANTHER classification system. Nat Protoc 2013; 8:1551-66.
17. Franceschini A, Szklarczyk D, Frankild S, Kuhn M, Simonovic M, Roth A, et al. STRING v9.1: protein-protein interaction networks, with increased coverage and integration. Nucleic Acids Res 2013; 41:D808-15.
18. Chen R, Pan S, Brentnall T A, Aebersold R. Proteomic profiling of pancreatic cancer for biomarker discovery. Mol Cell Proteomics 2005; 4:523-33.
19. Polanski M, Anderson N L. A list of candidate cancer biomarkers for targeted proteomics. Biomark Insights 2007; 1:1-48.

1. Method for determining a subject's probability to suffer from pancreatic cancer, comprising the steps of:
(i) providing a first sample from a subject whose probability to suffer from pancreatic cancer is to be determined, and determining the level of Platelet Glycoprotein V (GP5), or a peptide fragment thereof, in the first sample;
(ii) providing a second sample from a reference subject not suffering from pancreatic cancer, and determining the level of Platelet Glycoprotein V (GP5), or a peptide fragment thereof, in the second sample; and
(iii) comparing the level of Platelet Glycoprotein V (GP5), or a peptide fragment thereof, in said first and second sample;
wherein the steps (i) and (ii) can be carried out in any order, and wherein an increased level of GP5, or a peptide fragment thereof, in the first sample is indicative for an increased probability to suffer from pancreatic cancer.

2. The method according to claim 1, wherein the Platelet Glycoprotein V (GP5) comprises a polypeptide sequence which is at least 90% homologous, such as at least 95% homologous, or even homologous to SeqIDNo124, or wherein the peptide fragment thereof is at least 90% homologous, preferably at least 95% homologous or even homologous, to the corresponding part of SeqIDNo124.

3. The method according to claim 1, wherein said sample is a blood sample, such as a plasma or serum sample.

4. The method according to claim 1, wherein the level of Platelet Glycoprotein V (GP5), or a peptide fragment thereof, in the second sample used in step (iii) is an average value of at least two values from at least two different reference subjects, or the subject and the reference subject is the same person, but wherein the second sample in step (ii) was collected at a time when the subject did not suffer from pancreatic cancer.

5. (canceled)

6. Method according to claim 1, wherein the determination of the level of Platelet Glycoprotein V (GP5), or a peptide fragment thereof, in step (i) and step (ii) is conducted by ELISA, EIA, LC-MS, LC-MS/MS, gel-electrophoresis or comprising a step of treatment with a detectable moiety adapted to selectively bind to at least one of said at least one protein or polypeptide.

7. The method according to claim 1, wherein determination of the level of Platelet Glycoprotein V (GP5), or a peptide fragment thereof, in step (i) and (ii) is ELISA (enzyme-linked immunosorbent assay) or EIA (enzyme immunoassay) and the sample is as a plasma or serum sample.

8. The method according to claim 1, wherein a serum concentration of GP5, or a peptide fragment thereof, in the first sample at least 30% higher than of the second sample is indicative for an increased probability to suffer from pancreatic cancer, or a concentration of GP5 1.978 μg/L in said first sample is indicative for an increased probability to suffer from pancreatic cancer.

9. (canceled)

10. The method according to claim 1, wherein steps (i) and (ii) also comprises determining the level of at least one other protein or polypeptide in said first and second sample, said one protein or polypeptide being selected from the group consisting of CEA (Carcinoembryonic antigen), tumor marker CA 242, TAG-72 (Tumor-associated glycoprotein 72), HNRNPCL1, CA19-9, G7d, KAT2B, KIF20B, SMC1B and/or SPAG5 proteins, and
wherein step (iii) further comprises comparing the level of said at least one other protein or polypeptide in said first and second sample, and wherein an increased level of GP5, or a peptide fragment thereof, and said protein or polypeptide is indicative for an increased probability to suffer from pancreatic cancer.

11. The method according to claim 10, wherein the at least one protein or polypeptide is selected from the group consisting of HNRNPC1, CA19-9, G7d, KAT2B, KIF20B, SMC1B and/or SPAG5 proteins, and/or a group consisting of CEA (Carcinoembryonic antigen), tumor marker CA 242 and TAG-72 (Tumor-associated glycoprotein 72).

12. (canceled)

13. The method according to claim 10, wherein if the at least one protein or polypeptide is selected from the group consisting of Heterogeneous nuclear ribonucleoprotein C-like 1 (HNRNPCL1) and carbohydrate antigen 19-9 (CA19-9),
an increased level of GP5, or a peptide fragment thereof, and Heterogeneous nuclear ribonucleoprotein C-like 1 (HNRNPCL1) and/or carbohydrate antigen 19-9 (CA19-9) in the first sample compared to the second sample is indicative for an increased probability to suffer from pancreatic cancer, or
if the at least one protein or polypeptide is carbohydrate antigen 19-9 (CA19-9), a value of 2.729 or more for 0.562417*log (level GP5 in μg/L)+0.400120*log (level CA19-9 in μg/L) is indicative for an increased probability to suffer from pancreatic cancer.

14. (canceled)

15. The method according to claim 1, wherein the subject is in the perioperational phase after surgical removal of pancreatic cancer, and
said first sample is provided before surgical removal of pancreatic cancer and said second sample is provided during the perioperational phase after surgical removal of pancreatic cancer, or said first and second samples are provided from the subject at different times of the perioperational phase after surgical removal of pancreatic cancer, said second sample being provided after said first sample, and
wherein the sample concentration of GP5 in said samples is used to determine a subject's disease progression in the perioperational phase.

16. The method according to claim 15, wherein a decrease in concentration of GP5 in said second sample compared to the said first sample, is indicative of successful surgical removal or reduction in mass of pancreatic cancer tumor, and/or
an increase in serum concentration of GP5 in said second sample compared to the said first sample, is indicative of post-resection pancreatic cancer recurrence and pancreatic cancer disease progression.

17. (canceled)

18. The method according to claim 1, wherein step (i) and (ii) comprises:
treating said samples or a derivative thereof with a protease, said protease selectively cleaving at least a part of the peptide bonds of the comprising proteins and polypeptides thereof at the carboxylic acid side of lysine and arginine residues, to provide a plurality of polypeptide fragments, and
determining the level of at least one polypeptide fragment among the plurality of polypeptide fragments from the group consisting of SeqIDNo30, SeqIDNo31, SeqIDNo32 in said samples, wherein the fragment levels are directly correlating to the initial level of Platelet Glycoprotein V (GP5) in said samples.

19. (canceled)

20. Method for determining a subject's probability to suffer from pancreatic cancer, comprising the steps of:
(i) providing a sample from a subject whose probability to suffer from pancreatic cancer is to be determined, and determining the level of Platelet Glycoprotein V (GP5), or a peptide fragment thereof, in the sample; and
(ii) comparing the level of Platelet Glycoprotein V (GP5), or a peptide fragment thereof, with a reference value determined based on the level of Platelet Glycoprotein V (GP5), or a peptide fragment thereof, in samples from subjects known to suffer from pancreatic cancer and the level of Platelet Glycoprotein V (GP5), or a peptide fragment thereof, in samples from healthy subjects,
wherein a level of Platelet Glycoprotein V (GP5), or a peptide fragment thereof, above the reference value in said sample is indicative for an increased probability to suffer from pancreatic cancer.

21. The method according to claim 20, wherein the reference value is 1.978 μg/L, and
if a serum concentration of GP5, or a peptide fragment thereof, is more than 1.978 μg/ml, but less than 4.5 μg/L in said sample, this is indicative for an increased probability to suffer from pancreatic cancer stage I-II, or
if a serum concentration of GP5, or a peptide fragment thereof, is more than 4.5 μg/L in said sample, this is indicative for an increased probability to suffer from pancreatic cancer stage III-IV.

22-23. (canceled)

24. The method according to claim 20, wherein also the level of carbohydrate antigen 19-9 (CA19-9) is determined in the sample in step (i),
the reference value used in step (ii) being determined based on the level of Platelet Glycoprotein V (GP5), or a peptide fragment thereof, and the level of carbohydrate antigen 19-9 (CA19-9) in samples from subjects known to suffer from pancreatic cancer, and the level of Platelet Glycoprotein V (GP5), or a peptide fragment thereof, and the level of carbohydrate antigen 19-9 (CA19-9) in samples from healthy subjects, and
wherein a value of 2.729 or more for 0.562417*log (level GP5 in μg/L)+0.400120*log (level CA19-9 in μg/L) is indicative for an increased probability to suffer from pancreatic cancer.

25. The method according to claim 20, wherein the method of step (i) is ELISA (enzyme-linked immunosorbent assay) or EIA (enzyme immunoassay) and the sample is as a plasma or serum sample.

26. The method according to claim 21, wherein the indication is for an increased probability to suffer from pancreatic cancer stage I-II, and the method further comprises the steps of:
confirming the pancreatic cancer stage I-II prediction using a secondary clinical technique such as MRI (magnetic resonance imaging), CT scan, PET scan (positron emission tomography scan), Percutaneous transhepatic cholangiography (PTC), biopsy or laparoscopy,
establishing whether the pancreatic cancer appears surgically resectable, and
optionally where the pancreatic cancer appears resectable, surgically remove the tumor, preferably followed by chemotherapy or radiation treatment or both, or
wherein the indication is for an increased probability to suffer from pancreatic cancer stage III-IV, and the method further comprises the steps of:
confirming the pancreatic cancer stage III-IV prediction using a secondary clinical technique such as MRI (magnetic resonance imaging), CT scan, PET scan (positron emission tomography scan), Percutaneous transhepatic cholangiography (PTC), biopsy or laparoscopy,
establishing the extent of the spread of the tumor outside of the pancreas and whether the pancreatic cancer is surgically resectable, and
optionally where the pancreatic cancer appears resectable, surgically remove the tumor, preferably followed by chemotherapy or radiation treatment or both, or
optionally where the pancreatic cancer appears unresectable, avoid unnecessary explorative laparotomy and initiating either neoadjuvant therapy to downstage the tumor to allow subsequent resection or allow for life prolonging treatments such as chemotherapy with or without radiation therapy and/or alleviating symptoms form the pancreatic cancer through surgery, bile duct stents, opioid analgesics and antidepressants and counseling.

27-34. (canceled)

35. A kit for use in a method according to claim 1, said kit comprising means for measuring the level of Platelet Glycoprotein V (GP5), or a peptide fragment thereof, in a sample from a subject.

36. The kit according to claim 35, wherein said kit comprises a detecting antibody binding to Platelet Glycoprotein V (GP5), an enzyme-linked secondary antibody binding to the detecting antibody, and a substrate being converted by said enzyme to detectable form, and/or said kit further comprises a capture antibody binding to Platelet Glycoprotein V (GP5) and being bound to a surface, such as a microplate.

37. (canceled)


SEQUENCE LISTING (148 sequences)

SEQ ID NO: 1 - protein, 10 aa, human
LLVVYPWTQR

SEQ ID NO: 2 - protein, 17 aa, human
LSELHCDKLHVDPENFK

SEQ ID NO: 3 - protein, 6 aa, human
DICATK

SEQ ID NO: 4 - protein, 4 aa, human
DLLK

SEQ ID NO: 5 - protein, 6 aa, human
EVSVMR

SEQ ID NO: 6 - protein, 15 aa, human
KGSIHVSSAITEDQK

SEQ ID NO: 7 - protein, 4 aa, human
LSEK

SEQ ID NO: 8 - protein, 16 aa, human
NVTLDVQIQHVVEGKR

SEQ ID NO: 9 - protein, 14 aa, human
SSQDVSLDSNSNSK

SEQ ID NO: 10 - protein, 28 aa, human
VETEETHNYVGFEDIIDSLQDNVADIKK

SEQ ID NO: 11 - protein, 4 aa, human
LEER

SEQ ID NO: 12 - protein, 17 aa, human
QDADPGEVPGGSKESAR

SEQ ID NO: 13 - protein, 11 aa, human
SNSLPFVEGSR

SEQ ID NO: 14 - protein, 7 aa, human
TPTWVRR

SEQ ID NO: 15 - protein, 5 aa, human
LQHLK

SEQ ID NO: 16 - protein, 5 aa, human
QLTEK

SEQ ID NO: 17 - protein, 35 aa, human
TEAVREDLVPSESNAFLPSSVLWLSPSTALAADFR

SEQ ID NO: 18 - protein, 22 aa, human
VNHVDPEEEIVEHGAMEEREMR

SEQ ID NO: 19 - protein, 6 aa, human
EECAAK

SEQ ID NO: 20 - protein, 11 aa, human
EECAAKCEEDK

SEQ ID NO: 21 - protein, 11 aa, human
EQQCVIMAENR

SEQ ID NO: 22 - protein, 12 aa, human
EQQCVIMAENRK

SEQ ID NO: 23 - protein, 8 aa, human
KQLGAGSR

SEQ ID NO: 24 - protein, 9 aa, human
MRDAVLFEK

SEQ ID NO: 25 - protein, 24 aa, human
SGQGEPLDDYVNTQGPSLFSVTKK

SEQ ID NO: 26 - protein, 6 aa, human
SSIIIR

SEQ ID NO: 27 - protein, 8 aa, human
DSKFLLPK

SEQ ID NO: 28 - protein, 4 aa, human
LEEK

SEQ ID NO: 29 - protein, 6 aa, human
DVDNLK

SEQ ID NO: 30 - protein, 32 aa, human
ALFRNLSSLESVQLDHNQLETLPGDVFGALPR

SEQ ID NO: 31 - protein, 11 aa, human
AQPFPCPPACK

SEQ ID NO: 32 - protein, 15 aa, human
MVLLEQLFLDHNALR

SEQ ID NO: 33 - protein, 9 aa, human
AVLHGNLEK

SEQ ID NO: 34 - protein, 28 aa, human
AVQLRQEACATLLLQNGANPNITDFFGR

SEQ ID NO: 35 - protein, 13 aa, human
LLSHGTNIEECSK

SEQ ID NO: 36 - protein, 9 aa, human
RCELNLCDR

SEQ ID NO: 37 - protein, 21 aa, human
TALHLACATGQPEMVHLLVSR

SEQ ID NO: 38 - protein, 5 aa, human
TPLIK

SEQ ID NO: 39 - protein, 7 aa, human
WPTLMER

SEQ ID NO: 40 - protein, 11 aa, human
YLLLTYYDANK

SEQ ID NO: 41 - protein, 4 aa, human
EDLK

SEQ ID NO: 42 - protein, 6 aa, human
EEFYSR

SEQ ID NO: 43 - protein, 12 aa, human
FMPMDNLSGGEK

SEQ ID NO: 44 - protein, 16 aa, human
KAEENCLQTVNELMAK

SEQ ID NO: 45 - protein, 12 aa, human
LKYSQNELEMIK

SEQ ID NO: 46 - protein, 12 aa, human
RDIELEASQLDR

SEQ ID NO: 47 - protein, 13 aa, human
RFMPMDNLSGGEK

SEQ ID NO: 48 - protein, 12 aa, human
SGVISGGSSDLK

SEQ ID NO: 49 - protein, 9 aa, human
SVETLLNQK

SEQ ID NO: 50 - protein, 13 aa, human
CQIVNVAAEIFLK

SEQ ID NO: 51 - protein, 27 aa, human
DVNTSLGEVANETSENETLGDFSEQIK

SEQ ID NO: 52 - protein, 4 aa, human
EDLR

SEQ ID NO: 53 - protein, 18 aa, human
EETENASEPLGYESMASK

SEQ ID NO: 54 - protein, 11 aa, human
IGISAMYFYHK

SEQ ID NO: 55 - protein, 16 aa, human
KACIAQQTFIVPDLVK

SEQ ID NO: 56 - protein, 22 aa, human
LDSNSNCLSSVSAVEPTLMVIK

SEQ ID NO: 57 - protein, 4 aa, human
LQMR

SEQ ID NO: 58 - protein, 4 aa, human
LSEK

SEQ ID NO: 59 - protein, 4 aa, human
LWLK

SEQ ID NO: 60 - protein, 12 aa, human
QEITAVLEMKSR

SEQ ID NO: 61 - protein, 20 aa, human
SELMLQENQMIADGKEAETK

SEQ ID NO: 62 - protein, 5 aa, human
SLAEK

SEQ ID NO: 63 - protein, 11 aa, human
SNDDYQAAVER

SEQ ID NO: 64 - protein, 22 aa, human
SSEREAYSPLELLDNLSGADVR

SEQ ID NO: 65 - protein, 9 aa, human
TWPYYSCAR

SEQ ID NO: 66 - protein, 16 aa, human
TWPYYSCARISAWCWK

SEQ ID NO: 67 - protein, 16 aa, human
TWPYYSCARISAWCWK

SEQ ID NO: 68 - protein, 10 aa, human
YCKFHFNTLR

SEQ ID NO: 69 - protein, 4 aa, human
DELR

SEQ ID NO: 70 - protein, 15 aa, human
EEDEEGEDVVTSTGR

SEQ ID NO: 71 - protein, 9 aa, human
GSLSQEMAK

SEQ ID NO: 72 - protein, 12 aa, human
HAYVHKPYLYSK

SEQ ID NO: 73 - protein, 15 aa, human
KETEGTVTCTGAEGR

SEQ ID NO: 74 - protein, 4 aa, human
LEEK

SEQ ID NO: 75 - protein, 12 aa, human
LSESLHVVDENK

SEQ ID NO: 76 - protein, 4 aa, human
LVLK

SEQ ID NO: 77 - protein, 15 aa, human
TGDETELHSSEKGLK

SEQ ID NO: 78 - protein, 26 aa, human
VEDLSDAAIISTSTAECMPISASIDR

SEQ ID NO: 79 - protein, 7 aa, human
VEEAPVK

SEQ ID NO: 80 - protein, 40 aa, human
VLIISTSTTNDYTPQVSAITDVEGGLSDALRTEENMEGTR

SEQ ID NO: 81 - protein, 25 aa, human
VTTEEFEAPMPSAVSGDDSQLTASR

SEQ ID NO: 82 - protein, 16 aa, human
ANPGRDLLELLTEVNR

SEQ ID NO: 83 - protein, 24 aa, human
GLGYGCGGFGGLGYGYGCGCGSFR

SEQ ID NO: 84 - protein, 15 aa, human
QTALIAIPMSSQTPR

SEQ ID NO: 85 - protein, 17 aa, human
MASNVTNKMDPHSMNSR

SEQ ID NO: 86 - protein, 6 aa, human
QEVEVK

SEQ ID NO: 87 - protein, 11 aa, human
DSAWDPSQTMK

SEQ ID NO: 88 - protein, 12 aa, human
DTSVLTFTFCFK

SEQ ID NO: 89 - protein, 24 aa, human
NCDALLHHSAIPEDFLHIFLLLQK

SEQ ID NO: 90 - protein, 28 aa, human
EQDALWQGLELLQHGQAWFEDHLREAQR

SEQ ID NO: 91 - protein, 48 aa, human
LSCIDTTINEIINYGVSSFVIFVPIGLIFISYVLVISSILQIASAEGR

SEQ ID NO: 92 - protein, 49 aa, human
LSCIDTTINEIINYGVSSFVIFVPIGLIFISYVLVISSILQIASAEGRK

SEQ ID NO: 93 - protein, 27 aa, human
TFATCVSHLTVVIVHCGCASIAYLKPK

SEQ ID NO: 94 - protein, 7 aa, human
YTVIMSK

SEQ ID NO: 95 - protein, 7 aa, human
EFIPGEK

SEQ ID NO: 96 - protein, 16 aa, human
MNGSNMANTSPSVKSK

SEQ ID NO: 97 - protein, 12 aa, human
EAFTPEQLHLEK

SEQ ID NO: 98 - protein, 22 aa, human
LGYGGGYGGYGYGSGFGGYGYR

SEQ ID NO: 99 - protein, 6 aa, human
DESLYR

SEQ ID NO: 100 - protein, 5 aa, human
TPLLK

SEQ ID NO: 101 - protein, 12 aa, human
VPNFIGQYYKWK

SEQ ID NO: 102 - protein, 18 aa, human
GELAASANHGHSPCYPER

SEQ ID NO: 103 - protein, 10 aa, human
ALELEGAPAK

SEQ ID NO: 104 - protein, 26 aa, human
LLTLEQPQGDSMMTCEQAQLLANLAR

SEQ ID NO: 105 - protein, 19 aa, human
KCLFTHNIYLQDVQMVHPK

SEQ ID NO: 106 - protein, 4 aa, human
ELDK

SEQ ID NO: 107 - protein, 13 aa, human
FPPSLSETNKGLR

SEQ ID NO: 108 - protein, 17 aa, human
GLIPAGTQHSMIATTGK

SEQ ID NO: 109 - protein, 22 aa, human
GSETDTDSEIHESASDKDSLSK

SEQ ID NO: 110 - protein, 4 aa, human
LEEK

SEQ ID NO: 111 - protein, 5 aa, human
MEEQR

SEQ ID NO: 112 - protein, 30 aa, human
DIQMTQSPSPLSASVGDSVTITCQASQDIR

SEQ ID NO: 113 - protein, 16 aa, human
FLIYDAENLEIGVPSR

SEQ ID NO: 114 - protein, 13 aa, human
MSYNCCSGNFSSR

SEQ ID NO: 115 - protein, 7 aa, human
LGHNELK

SEQ ID NO: 116 - protein, 11 aa, human
LGHNELKECLK

SEQ ID NO: 117 - protein, 147 aa, human
MVHFTAEEKAAVTSLWSKMNVEEAGGEALGRLLVVYPWTQRFFDSFGNLSSPSAILGNPKVKAHGKKVLTSFGDAIKNMDNLKPAFAKLSELHCDKLHVDPENFKLLGNVMVIILATHFGKEFTPEVQAAWQKLVSAVAIALAHKYH

SEQ ID NO: 118 - protein, 3580 aa, human
MESNFNQEGVPRPSYVFSADPIARPSEINFDGIKLDLSHEFSLVAPNTEANSFESKDYLQVCLRIRPFTQSEKELESEGCVHILDSQTVVLKEPQCILGRLSEKSSGQMAQKFSFSKVFGPATTQKEFFQGCIMQPVKDLLKGQSRLIFTYGLTNSGKTYTFQGTEENIGILPRTLNVLFDSLQERLYTKMNLKPHRSREYLRLSSEQEKEEIASKSALLRQIKEVTVHNDSDDTLYGSLTNSLNISEFEESIKDYEQANLNMANSIKFSVWVSFFEIYNEYIYDLFVPVSSKFQKRKMLRLSQDVKGYSFIKDLQWIQVSDSKEAYRLLKLGIKHQSVAFTKLNNASSRSHSIFTVKILQIEDSEMSRVIRVSELSLCDLAGSERTMKTQNEGERLRETGNINTSLLTLGKCINVLKNSEKSKFQQHVPFRESKLTHYFQSFFNGKGKICMIVNISQCYLAYDETLNVLKFSAIAQKVCVPDTLNSSQEKLFGPVKSSQDVSLDSNSNSKILNVKRATISWENSLEDLMEDEDLVEELENAEETQNVETKLLDEDLDKTLEENKAFISHEEKRKLLDLIEDLKKKLINEKKEKLTLEFKIREEVTQEFTQYWAQREADFKETLLQEREILEENAERRLAIFKDLVGKCDTREEAAKDICATKVETEETHNYVGFEDIIDSLQDNVADIKKQAEIAHLYIASLPDPQEATACLELKFNQIKAELAKTKGELIKTKEELKKRENESDSLIQELETSNKKIITQNQRIKELINIIDQKEDTINEFQNLKSHMENTFKCNDKADTSSLIINNKLICNETVEVPKDSKSKICSERKRVNENELQQDEPPAKKGSIHVSSAITEDQKKSEEVRPNIAEIEDIRVLQENNEGLRAFLLTIENELKNEKEEKAELNKQIVHFQQELSLSEKKNLTLSKEVQQIQSNYDIAIAELHVQKSKNQEQEEKIMKLSNEIETATRSITNNVSQIKLMHTKIDELRTLDSVSQISNIDLLNLRDLSNGSEEDNLPNTQLDLLGNDYLVSKQVKEYRIQEPNRENSFHSSIEAIWEECKEIVKASSKKSHQIEELEQQIEKLQAEVKGYKDENNRLKEKEHKNQDDLLKEKETLIQQLKEELQEKNVTLDVQIQHVVEGKRALSELTQGVTCYKAKIKELETILETQKVECSHSAKLEQDILEKESIILKLERNLKEFQEHLQDSVKNTKDLNVKELKLKEEITQLTNNLQDMKHLLQLKEEEEETNRQETEKLKEELSASSARTQNLKADLQRKEEDYADLKEKLTDAKKQIKQVQKEVSVMRDEDKLLRIKINELEKKKNQCSQELDMKQRTIQQLKEQLNNQKVEEAIQQYERACKDLNVKEKIIEDMRMTLEEQEQTQVEQDQVLEAKLEEVERLATELEKWKEKCNDLETKNNQRSNKEHENNTDVLGKLTNLQDELQESEQKYNADRKKWLEEKMMLITQAKEAENIRNKEMKKYAEDRERFFKQQNEMEILTAQLTEKDSDLQKWREERDQLVAALEIQLKALISSNVQKDNEIEQLKRIISETSKIETQIMDIKPKRISSADPDKLQTEPLSTSFEISRNKIEDGSVVLDSCEVSTENDQSTRFPKPELEIQFTPLQPNKMAVKHPGCTTPVTVKIPKARKRKSNEMEEDLVKCENKKNATPRTNLKFPISDDRNSSVKKEQKVAIRPSSKKTYSLRSQASIIGVNLATKKKEGTLQKFGDFLQHSPSILQSKAKKIIETMSSSKLSNVEASKENVSQPKRAKRKLYTSEISSPIDISGQVILMDQKMKESDHQIIKRRLRTKTAKVCLRIRPFTQSEKELESEGCVHILDSQTVVLKEPQCILGRLSEKSSGQMAQKFSFSKVFGPATTQKEFFQGCIMQPVKDLLKGQSRLIFTYGLTNSGKTYTFQGTEENIGILPRTLNVLFDSLQERLYTKMNLKPHRSREYLRLSSEQEKEEIASKSALLRQIKEVTVHNDSDDTLYGSLTNSLNISEFEESIKDYEQANLNMANSIKFSVWVSFFEIYNEYIYDLFVPVSSKFQKRKMLRLSQDVKGYSFIKDLQWIQVSDSKEAYRLLKLGIKHQSVAFTKLNNASSRSHSIFTVKILQIEDSEMSRVIRVSELSLCDLAGSERTMKTQNEGERLRETGNINTSLLTLGKCINVLKNSEKSKFQQHVPFRESKLTHYFQSFFNGKGKICMIVNISQCYLAYDETLNVLKFSAIAQKVCVPDTLNSSQEKLFGPVKSSQDVSLDSNSNSKILNVKRATISWENSLEDLMEDEDLVEELENAEETQNVETKLLDEDLDKTLEENKAFISHEEKRKLLDLIEDLKKKLINEKKEKLTLEFKIREEVTQEFTQYWAQREADFKETLLQEREILEENAERRLAIFKDLVGKCDTREEAAKDICATKVETEETHNYVGFEDIIDSLQDNVADIKKQAEIAHLYIASLPDPQEATACLELKFNQIKAELAKTKGELIKTKEELKKRENESDSLIQELETSNKKIITQNQRIKELINIIDQKEDTINEFQNLKSHMENTFKCNDKADTSSLIINNKLICNETVEVPKDSKSKICSERKRVNENELQQDEPPAKKGSIHVSSAITEDQKKSEEVRPNIAEIEDIRVLQENNEGLRAFLLTIENELKNEKEEKAELNKQIVHFQQELSLSEKKNLTLSKEVQQIQSNYDIAIAELHVQKSKNQEQEEKIMKLSNEIETATRSITNNVSQIKLMHTKIDELRTLDSVSQISNIDLLNLRDLSNGSEEDNLPNTQLDLLGNDYLVSKQVKEYRIQEPNRENSFHSSIEAIWEECKEIVKASSKKSHQIEELEQQIEKLQAEVKGYKDENNRLKEKEHKNQDDLLKEKETLIQQLKEELQEKNVTLDVQIQHVVEGKRALSELTQGVTCYKAKIKELETILETQKVECSHSAKLEQDILEKESIILKLERNLKEFQEHLQDSVKNTKDLNVKELKLKEEITQLTNNLQDMKHLLQLKEEEEETNRQETEKLKEELSASSARTQNLKADLQRKEEDYADLKEKLTDAKKQIKQVQKEVSVMRDEDKLLRIKINELEKKKNQCSQELDMKQRTIQQLKEQLNNQKVEEAIQQYERACKDLNVKEKIIEDMRMTLEEQEQTQVEQDQVLEAKLEEVERLATELEKWKEKCNDLETKNNQRSNKEHENNTDVLGKLTNLQDELQESEQKYNADRKKWLEEKMMLITQAKEAENIRNKEMKKYAEDRERFFKQQNEMEILTAQLTEKDSDLQKWREERDQLVAALEIQLKALISSNVQKDNEIEQLKRIISETSKIETQIMDIKPKRISSADPDKLQTEPLSTSFEISRNKIEDGSVVLDSCEVSTENDQSTRFPKPELEIQFTPLQPNKMAVKHPGCTTPVTVKIPKARKRKSNEMEEDLVKCENKKNATPRTNLKFPISDDRNSSVKKEQKVAIRPSSKKTYSLRSQASIIGVNLATKKKEGTLQKFGDFLQHSPSILQSKAKKIIETMSSSKLSNVEASKENVSQPKRAKRKLYTSEISSPIDISGQVILMDQKMKESDHQIIKRRLRTKTAK

SEQ ID NO: 119 - protein, 1677 aa, human
MEVPEPTCPAPPARDQPAPTPGPPGAPGGQASPHLTLGPVLLPPEQGLAPPTVFLKALPIPLYHTVPPGGLQPRAPLVTGSLDGGNVPFILSPVLQPEGPGPTQVGKPAAPTLTVNIVGTLPVLSPGLGPTLGSPGKVRNAGKYLCPHCGRDCLKPSVLEKHIRSHTGERPFPCATCGIAFKTQSNLYKHRRTQTHLNNSRLSSESEGAGGGLLEEGDKAGEPPRPEGRGESRCQGMHEGASERPLSPGAHVPLLAKNLDVRTEAAPCPGSAFADREAPWDSAPMASPGLPAASTQPWRKLPEQKSPTAGKPCALQRQQATAAEKPWDAKAPEGRLRKCESTDSGYLSRSDSAEQPHAPCSPLHSLSEHSAESEGEGGPGPGPGVAGAEPGAREAGLELEKKRLEERIAQLISHNQAVVDDAQLDNVRPRKTGLSKQGSIDLPTPYTYKDSFHFDIRALEPGRRRAPGPVRSTWTPPDKSRPLFFHSVPTQLSTTVECVPVTRSNSLPFVEGSRTWLEPREPRDPWSRTQKPLSPRPGPARLGCRSGLSSTDVPSGHPRALVRQAAVEDLPGTPIGDALVPAEDTDAKRTAAREAMAGKGRAGGRKCGQRRLKMFSQEKWQVYGDETFKRIYQKMKASPHGGKKAREVGMGSGAELGFPLQKEAAGSSGTVPTQDRRTPVHEDISAGATPEPWGNPPALEASLVTEPTKHGETVARRGDSDRPRVEEAVSSPALGGRDSPCSGSRSPLVSPNGRLELGWQMPPAPGPLKGGDVEAPRPVWPDPKLEGGARGVGDVQETCLWAQTVLRWPSRGSGEDKLPSERKKLKVEDLHSWKQPEPVSAETPGGPTQPASLSSQKQDADPGEVPGGSKESARQVGEPLESSGASLAAASVALKRVGPRDKATPLHPAAPAPAEHPSLATPPQAPRVLSALADNAFSPKYLLRLPQAETPLPLPIPWGPRHSQDSLCSSGWPEERASFVGSGLGTPLSPSPASGPSPGEADSILEDPSCSRPQDGRKGAQLGGDKGDRMATSRPAARELPISAPGAPREATSSPPTPTCEAHLVQDMEGDSHRIHRLCMGSTLARARLSGDVLNPWVPNWELGEPPGNAPEDPSSGPLVGPDPCSPLQPGSFLTALTRPQGVPPGWPELALSSHSGTSRSHSTRSPHSTQNPFPSLKAEPRLTWCCLSRSVPLPAEQKAKAASVYLAVHFPGSSLRDEGPNGPPGSNGGWTWTSPGEGGPAQMSKFSYPTVPGVMPQHQVSEPEWKKGLPWRAKMSRGNSKQRKLKINPKRYKGNFLQSCVQLRASRLRTPTWVRRRSRHPPALEGLKPCRTPGQTSSEIAGLNLQEEPSCATSESPPCCGKEEKKEGDCRQTLGTLSLGTSSRIVREMDKRTVKDISPSAGEHGDCTTHSTAATSGLSLQSDTCLAVVNDVPLPPGKGLDLGLLETQLLASQDSVSTDPKPYIFSDAQRPSSFGSKGTFPHHDIATSVAAVCISLPVRTDHIAQEIHSAESRDHSQTAGRTLTSSSPDSKVTEEGRAQTLLPGRPSSGQRISDSVPLESTEKTHLEIPASGPSSASSHHKEGRHKTFFPSRGQYGCGEMTVPCPSLGSDGRKRQVSGLITRKDSVVPSKPEQPIEIPEAPSKSLKKRSLEGMRKQTRVEFSDTSSDDEDRLVIEI

SEQ ID NO: 120 - protein, 1193 aa, human
MWRVKKLSLSLSPSPQTGKPSMRTPLRELTLQPGALTNSGKRSPACSSLTPSLCKLGLQEGSNNSSPVDFVNNKRTDLSSEHFSHSSKWLETCQHESDEQPLDPIPQISSTPKTSEEAVDPLGNYMVKTIVLVPSPLGQQQDMIFEARLDTMAETNSISLNGPLRTDDLVREEVAPCMGDRFSEVAAVSEKPIFQESPSHLLEESPPNPCSEQLHCSKESLSSRTEAVREDLVPSESNAFLPSSVLWLSPSTALAADFRVNHVDPEEEIVEHGAMEEREMRFPTHPKESETEDQALVSSVEDILSTCLTPNLVEMESQEAPGPAVEDVGRILGSDTESWMSPLAWLEKGVNTSVMLENLRQSLSLPSMLRDAAIGTTPFSTCSVGTWFTPSAPQEKSTNTSQTGLVGTKHSTSETEQLLCGRPPDLTALSRHDLEDNLLSSLVILEVLSRQLRDWKSQLAVPHPETQDSSTQTDTSHSGITNKLQHLKESHEMGQALQQARNVMQSWVLISKELISLLHLSLLHLEEDKTTVSQESRRAETLVCCCFDLLKKLRAKLQSLKAEREEARHREEMALRGKDAAEIVLEAFCAHASQRISQLEQDLASMREFRGLLKDAQTQLVGLHAKQEELVQQTVSLTSTLQQDWRSMQLDYTTWTALLSRSRQLTEKLTVKSQQALQERDVAIEEKQEVSRVLEQVSAQLEECKGQTEQLELENSRLATDLRAQLQILANMDSQLKELQSQHTHCAQDLAMKDELLCQLTQSNEEQAAQWQKEEMALKHMQAELQQQQAVLAKEVRDLKETLEFADQENQVAHLELGQVECQLKTTLEVLRERSLQCENLKDTVENLTAKLASTIADNQEQDLEKTRQYSQKLGLLTEQLQSLTLFLQTKLKEKTEQETLLLSTACPPTQEHPLPNDRTFLGSILTAVADEEPESTPVPLLGSDKSAFTRVASMVSLQPAETPGMEESLAEMSIMTTELQSLCSLLQESKEEAIRTLQRKICELQARLQAQEEQHQEVQKAKEADIEKLNQALCLRYKNEKELQEVIQQQNEKILEQIDKSGELISLREEVTHLTRSLRRAETETKVLQEALAGQLDSNCQPMATNWIQEKVWLSQEVDKLRVMFLEMKNEKEKLMIKFQSHRNILEENLRRSDKELEKLDDIVQHIYKTLLSIPEVVRGCKELQGLLEFLS

SEQ ID NO: 121 - protein, 96 aa, human
MEHKEVVLLLLLFLKSGQGEPLDDYVNTQGPSLFSVTKKQLGAGSREECAAKCEEDKEFTCRAFQYHSKEQQCVIMAENRKSSIIIRMRDAVLFEK

SEQ ID NO: 122 - protein, 1265 aa, human
MKVRLLRQLSAAAKVKAPSGLQGPPQAHQFISLLLEEYGALCQAARSISTFLGTLENEHLKKFQVTWELHNKHLFENLVFSEPLLQSNLPALVSQIRLGTTTHDTCSEDTYSTLLQRYQRSEEELRRVAEEWLECQKRIDAYVDEQMTMKTKQRMLTEDWELFKQRRFIEEQLTNKKAVTGENNFTDTMRHMLSSRLSMPDCPNCNYRRRCACDDCSLSHILTCGIMDPPVTDDIHIHQLPLQVDPAPDYLAERSPPSVSSASSGSGSSSPITIQQHPRLILTDSGSAPTFCSDDEDVAPLSAKFADIYPLSNYDDTEVVANMNGIHSELNGGGENMALKDESPQISSTSSSSSEADDEEADGESSGEPPGAPKEDGVLGSRSPRTEESKADSPPPSYPTQQAEQAPNTCECHVCKQEASGLTPSAMTAGALPPGHQFLSPEKPTHPALHLYPHIHGHVPLHTVPHLPRPLIHPTLYATPPFTHSKALPPAPVQNHTNKHQVFNASLQDHIYPSCFGNTPEWNSSKFISLWGSEVMNDKNWNPGTFLPDTISGSEILGPTLSETRPEALPPPSSNETPAVSDSKEKKNAAKKKCLYNFQDAFMEANKVVMATSSATSSVSCTATTVQSSNSQFRVSSKRPPSVGDVFHGISKEDHRHSAPAAPRNSPTGLAPLPALSPAALSPAALSPASTPHLANLAAPSFPKTATTTPGFVDTRKSFCPAPLPPATDGSISAPPSVCSDPDCEGHRCENGVYDPQQDDGDESADEDSCSEHSSSTSTSTNQKEGKYCDCCYCEFFGHGGPPAAPTSRNYAEMREKLRLRLTKRKEEQPKKMDQISERESVVDHRRVEDLLQFINSSETKPVSSTRAAKRARHKQRKLEEKARLEAEARAREHLHLQEEQRRREEEEDEEEEEDRFKEEFQRLQELQKLRAVKKKKKERPSKDCPKLDMLTRNFQAATESVPNSGNIHNGSLEQTEEPETSSHSPSRHMNHSEPRPGLGADGDAADPVDTRDSKFLLPKEVNGKQHEPLSFFFDIMQHHKEGNGKQKLRQTSKASSEPARRPTEPPKATEGQSKPRAQTESKAKVVDLMSITEQKREERKVNSNNNNKKQLNHIKDEKSNPTPMEPTSPGEHQQNSKLVLAESPQPKGKNKKNKKKKGDRVNNSIDGVSLLLPSLGYNGAILAHCNLRLPGSSDCAASASQVVGITDDVFLPKDIDLDSVDMDETEREVEYFKRFCLDSARQTRQRLSINWSNFSLKKATFAAH

SEQ ID NO: 123 - protein, 259 aa, human
MKDVDNLKSIKEEWVCETGSDNQPLGNNQQSNCEYFVDSLFEEAQKVSSKCVSPAEQKKQVDVNIKLWKNGFTVNDDFRSYSDGASQQFLNSIKKGELPSELQGIFDKEEVDVKVEDKKNEICLSTKPVFQPFSGQGHRLGSATPKIVSKAKNIEVENKNNLSAVPLNNLEPITNIQIWLANGKRIVQKFNITHRVSHIKDFIEKYQGSQRSPPFSLATALPVLRLLDETLTLEEADLQNAVIIQRLQKTASFRELSEH

SEQ ID NO: 124 - protein, 560 aa, human
MLRGTLLCAVLGLLRAQPFPCPPACKCVFRDAAQCSGGDVARISALGLPTNLTHILLFGMGRGVLQSQSFSGMTVLQRLMISDSHISAVAPGTFSDLIKLKTLRLSRNKITHLPGALLDKMVLLEQLFLDHNALRGIDQNMFQKLVNLQELALNQNQLDFLPASLFTNLENLKLLDLSGNNLTHLPKGLLGAQAKLERLLLHSNRLVSLDSGLLNSLGALTELQFHRNHIRSIAPGAFDRLPNLSSLTLSRNHLAFLPSALFLHSHNLTLLTLFENPLAELPGVLFGEMGGLQELWLNRTQLRTLPAAAFRNLSRLRYLGVTLSPRLSALPQGAFQGLGELQVLALHSNGLTALPDGLLRGLGKLRQVSLRRNRLRALPRALFRNLSSLESVQLDHNQLETLPGDVFGALPRLTEVLLGHNSWRCDCGLGPFLGWLRQHLGLVGGEEPPRCAGPGAHAGLPLWALPGGDAECPGPRGPPPRPAADSSSEAPVHPALAPNSSEPWVWAQPVTTGKGQDHSPFWGFYFLLLAVQAMITVIIVFAMIKIGQLFRKLIRERALG

SEQ ID NO: 125 - protein, 1941 aa, human
MEDGKRERWPTLMERLCSDGFAFPQYPIKPYHLKRIHRAVLHGNLEKLKYLLLTYYDANKRDRKERTALHLACATGQPEMVHLLVSRRCELNLCDREDRTPLIKAVQLRQEACATLLLQNGANPNITDFFGRTALHYAVYNEDTSMIEKLLSHGTNIEECSKCEYQPLLFAVSRRKVKMVEFLLKKKANVNAIDYLGRSALIHAVTLGEKDIVILLLQHNIDVLSRDAFRKIAGDYAIEAKNRVIFDLIYEYERKRYEDLPINSNPVSSQKQPALKATSGKEDSISNIATEIKDGQKSGTVSSQKQPALKDTSDKDDSVSNTATEIKDEQKSGTVLPAVEQCLNRSLYRPDAVAQPVTENEFSLESEIISKLYIPKRKIISPRSIKDVLPPVEEAVDRCLYLLDRFAQPVTKDKFALESENISEPYFTNRRTISQQSAENLDAACGIDKTENGNMFEDQNVDKEGKALPATGQKANVSPEQPPLFTHTVKDRDHISTRFLGGMDSLTSSEESSERPPLSTLTLKEADPSSKAAMRRKDSPPPGKVSSQKQPAEKATSDDKDSVSNIATEIKEGPISGTVSSQKQPAEKATSDEKDSVSNIATEIKKGQQSGTVSPQKQSAWKVIFKKKVSLLNIATRIMGGGKSGTVSSQKQPASKATSDKTDSALNIATEIKDGLQCGTVSSQKQPALKATTDEEDSVSNIATEIKDGEKSGTVSSQKQPALKATTDEEDSVSNIATEIKDGEKSGTVSSQKQPALKATTDEKDSVSNIATEIKDGEKSGTVSSQKPPALTATSDEEGSVLSIARENKDGEKSRTVSSRKKPALKATSDEKDSFSNITRGKKDGEISRKVSSQKPPTLKGTSDEEDSVLGIARENKDGEKSRTVSSEKPPGLKASSAEKDSVLNIARGKKDGEKTKRVSSRKKPSLEATSDEKDSFSNITREKKDGEISRKVSSQKPPALKGTSDEEDSVLGIARENKDGEKSRTVSSEKPPGLKATSDEKDSVLNIARGKKDGEKTRTVSSQKPPTLKATSDEEDSVLSIARENKDGEKSRTVSSEKPSGLKATSAEKDSVLNIARGKKYGEKTKRVSSRKKPALKATSDEKDSVLYIAREKKDGEKSRTVSSPKQPALKAICDKEDSVPNMATEKKDEQISGTVSCQKQPALKATSDKKDSVSNIPTEIKDGQQSGTVSSQKQPAWKATSVKKDSVSNIATEIKDGQIRGTGILEYTFNVMFDQIEEKFTSLNKSAGVSPQKQSAQKVIFKKKVSLLNIATRITGGWKSGTEYPENLPTLKATIENKNSVLNTATKMKDVQTSTPAEQDLEMASEGEQKRLEEYENNQPQVKNQIHSRDDLDDIIQSSQTVSEDGDSLCCNCKNVILLIDQHEMKCKDCVHLLKIKNTFCLWKRLIKLKDNHCEQLRVKIRKLKNKASVLQKRISEKEEIKSQLKHEILELEKELCSLRFAIQQEKKKRRNVEEVHQKVREKLRITEEQYRIEADVTKPIKPALKSAEVELKTGGNNSNQVSETDEKEDLLHENRLMQDEIARLRLEKDTIKNQNLEKKYLKDFEIVKRKHEDLQKALKRNGETLAKTIACYSGQLAALTDENTTLRSKLEKQRESRQRLETEMQSYHCRLNAARCDHDQSHSSKRDQELAFQGTVDKCRHLQENLNSHVLILSLQLSKAESKSRVLKTELHYTGEALKEKALVFEHVQSELKQKQSQMKDIEKMYKSGYNTMEKCIEKQERFCQLKKQNMLLQQQLDDARNKADNQEKAILNIQARCDARVQNLQAECRKHRLLLEEDNKMLVNELNHSKEKECQYEKEKAEREVAVRQLQQKRDDVLNKGSATKALLDASSRHCTYLENGMQDSRKKLDQMRSQFQEIQDQLTATIRCTKEMEGDTQKLEVEHVMMRKIIKKQDDQIERLEKILQHSSLMLQVFES

SEQ ID NO: 126 - protein, 1235 aa, human
MAHLELLLVENFKSWRGRQVIGPFRRFTCIIGPNGSGKSNVMDALSFVMGEKIANLRVKNIQELIHGAHIGKPISSSASVKIIYVEESGEEKTFARIIRGGCSEFRFNDNLVSRSVYIAELEKIGIIVKAQNCLVFQGTVESISVKKPKERTQFFEEISTSGELIGEYEEKKRKLQKAEEDAQFNFNKKKNIAAERRQAKLEKEEAERYQSLLEELKMNKIQLQLFQLYHNEKKIHLLNTKLEHVNRDLSVKRESLSHHENIVKARKKEHGMLTRQLQQTEKELKSVETLLNQKRPQYIKAKENTSHHLKKLDVAKKSIKDSEKQCSKQEDDIKALETELADLDAAWRSFEKQIEEEILHKKRDIELEASQLDRYKELKEQVRKKVATMTQQLEKLQWEQKTDEERLAFEKRRHGEVQGNLKQIKEQIEDHKKRIEKLEEYTKTCMDCLKEKKQQEETLVDEIEKTKSRMSEFNEELNLIRSELQNAGIDTHEGKRQQKRAEVLEHLKRLYPDSVFGRLFDLCHPIHKKYQLAVTKVFGRFITAIVVASEKVAKDCIRFLKEERAEPETFLALDYLDIKPINERLRELKGCKMVIDVIKTQFPQLKKVIQFVCGNGLVCETMEEARHIALSGPERQKTVALDGTLFLKSGVISGGSSDLKYKARCWDEKELKNLRDRRSQKIQELKGLMKTLRKETDLKQIQTLIQGTQTRLKYSQNELEMIKKKHLVAFYQEQSQLQSELLNIESQCIMLSEGIKERQRRIKEFQEKIDKVEDDIFQHFCEEIGVENIREFENKHVKRQQEIDQKRYFYKKMLTRLNVQLEYSRSHLKKKLNKINTLKETIQKGSEDIDHLKKAEENCLQTVNELMAKQQQLKDIRVTQNSSAEKVQTQIEEERKKFLAVDREVGKLQKEVVSIQTSLEQKRLEKHNLLLDCKVQDIEIILLSGSLDDIIEVEMGTEAESTQATIDIYEKEEAFEIDYSSLKEDLKALQSDQEIEAHLRLLLQQVASQEDILLKTAAPNLRALENLKTVRDKFQESTDAFEASRKEARLCRQEFEQVKKRRYDLFTQCFEHVSISIDQIYKKLCRNNSAQAFLSPENPEEPYLEGISYNCVAPGKRFMPMDNLSGGEKCVAALALLFAVHSFRPAPFFVLDEVDAALDNTNIGKVSSYIKEQTQDQFQMIVISLKEEFYSRADALIGIYPEYDDCMFSRVLTLDLSQYPDTEGQESSKRHGESR

SEQ ID NO: 127 - protein, 1692 aa, human
MRRPPPLGPTTASGPEGNVRNLQKRQAPGPGAAGGCGPEAGGCRENKQKRRMVARATPGRGEVESDKSVAASGAGKAARRQVEGRRGPVSPSDSSDPRGLEAAKEAELPLQTERHTKEKRKVTEASSDDPQPGLDLVRKESLTSSESFQTVECLQSLGKESIIEGIKRRIRNKKLKSLENPPLKITENEATQNIKVEFQDELYKNTPKYSCNILSPEVENNSVLKLRDCNCFPHSKGCNDENNLPYKPDGGCMHVAENFSKKENLRSLAEKSDTNSIPQLLQTEENVMGVNKLLPEESDLYQSKTNGLLSCLQHEKNKYSIEESSVGRKPRKRMKLSEKADETVTEMNFSNEYNKSELMLQENQMIADGKEAETKSPLNVLRKVSHNTVSLMDHLLSVPETVEKETSSEHHVNAVFQKTIEPLLKEETENASEPLGYESMASKEDFKSMKSFIGKSPNEYHIERRSSREDLRSASEELKLSCQRTIPMTGKRTWPYYSCARISAWCWKKASLPESSYFLRGSQESCRQVDVPKHQTNQTHLTDSKLLLQSSLTETNTESSSKEKLDSNSNCLSSVSAVEPTLMVIKEPIIKDDKKIKSEELSRRGSEVISNTTEDTQLTSETQSLTGNKKKARGNLTKLNLTATSKDGQEANNSAGKTIHRKACIAQQTFIVPDLVKILNTGRLTNFKIPLLKNKSEKRKEVNAKSSEREAYSPLELLDNLSGADVRQNRSKENVSMMMLGPQTLSIRNSVTPVQASSDSFYNKKSYSISPSFTKQGNNSKPSNHVSEPGNIVSNKEVASLTVENNAFSCDPGYVEKSPSFCCNEQETFRPVSSEVRGRKITKNFSEVGFPDILKAYEDDVLLIDVIQDDPDLFGVSNEGELSFTSEVPKISQEPNVAGEHQSTDSKYMETPVKKEPSDDLRELPVLDCGWIKPDICASNSAESEIKRDPKDVNTSLGEVANETSENETLGDFSEQIKGSDLDEKHRFTDKVITKEEKENIYEVCKSKDSRNADFMVGECQFAVPVPKPLCLLVPPLNLSGRQEDTILNTWMNDFRFLGKHSVLKLQNPETCEIFKREKNVGVFQKSLGLMIPYKYCKFHFNTLRGCERPLCKFAHVPEQGDEKVCMDVFKKYININELCLLQRAVNIFMEYYRKFPPGVYFDLQVLNDLLNSLLKHCLLKEVFQIVNLSIMVKMLPSLKILLNIFEYVATMKLRNAVPALIDIFCKLVEAGMVLDPEHFNYIVKLLYQVQASKQEITAVLEMKSRLQMRRFKKNWKCDLDSALNKLEHCKEKGDWTKLGKLYINVKMGCEKFADFQTFCACIAETLTKNYEDERPDIPFCEFAETVSKDPQNSKVDKGVLGRIGISAMYFYHKLLQWSKGRKVLEKLYELKIHFTSLKGLIGPEKLASRCQIVNVAAEIFLKSGSLDGAIWVMRESEWIINTPLWPCDRLDVLNRHNLLCTIAHEILAKSLYRQTFEVLQNLPGFQNSQETVEVSQYSLLFNKLLGSCIESSSLGMSSSVAEFMISKSIPIDFSFLRRLITSLGRSRLWLKARAHYKSALSLGCYPPLEGNLYRKLLLIPSYLSEIEMLLAIEIFMVSNASSIQSPGTSTQILQIVLKRCEDNQSRSNDDYQAAVERLIMAARISDPKLFVKHMTVNVNKEQVYSLEHCSALKWLKENMKWAGKVWLFSNH

SEQ ID NO: 128 - protein, 3051 aa, human
MATNPQPQPPPPAPPPPPPQPQPQPPPPPPGPGAGPGAGGAGGAGAGAGDPQLVAMIVNHLKSQGLFDQFRRDCLADVDTKPAYQNLRQRVDNFVANHLATHTWSPHLNKNQLRNNIRQQVLKSGMLESGIDRIISQVVDPKINHTFRPQVEKAVHEFLATLNHKEEGSGNTAPDDEKPDTSLITQGVPTPGPSANVANDAMSILETITSLNQEASAARASTETSNAKTSERASKKLPSQPTTDTSTDKERTSEDMADKEKSTADSGGEGLETAPKSEEFSDLPCPVEEIKNYTKEHNNLILLNKDVQQESSEQKNKSTDKGEKKPDSNEKGERKKEKKEKTEKKFDHSKKSEDTQKVKDEKQAKEKEVESLKLPSEKNSNKAKTVEGTKEDFSLIDSDVDGLTDITVSSVHTSDLSSFEEDTEEEVVTSDSMEEGEITSDDEEKNKQNKTKTQTSDSSEGKTKSVRHAYVHKPYLYSKYYSDSDDELTVEQRRQSIAKEKEERLLRRQINREKLEEKRKQKAEKTKSSKTKGQGRSSVDLEESSTKSLEPKAARIKEVLKERKVLEKKVALSKKRKKDSRNVEENSKKKQQYEEDSKETLKTSEHCEKEKISSSKELKHVHAKSEPSKPARRLSESLHVVDENKNESKLEREHKRRTSTPVIMEGVQEETDTRDVKRQVERSEICTEEPQKQKSTLKNEKHLKKDDSETPHLKSLLKKEVKSSKEKPEREKTPSEDKLSVKHKYKGDCMHKTGDETELHSSEKGLKVEENIQKQSQQTKLSSDDKTERKSKHRNERKLSVLGKDGKPVSEYIIKTDENVRKENNKKERRLSAEKTKAEHKSRRSSDSKIQKDSLGSKQHGITLQRRSESYSEDKCDMDSTNMDSNLKPEEVVHKEKRRTKSLLEEKLVLKSKSKTQGKQVKVVETELQEGATKQATTPKPDKEKNTEENDSEKQRKSKVEDKPFEETGVEPVLETASSSAHSTQKDSSHRAKLPLAKEKYKSDKDSTSTRLERKLSDGHKSRSLKHSSKDIKKKDENKSDDKDGKEVDSSHEKARGNSSLMEKKLSRRLCENRRGSLSQEMAKGEEKLAANTLSTPSGSSLQRPKKSGDMTLIPEQEPMEIDSEPGVENVFEVSKTQDNRNNNSQQDIDSENMKQKTSATVQKDELRTCTADSKATAPAYKPGRGTGVNSNSEKHADHRSTLTKKMHIQSAVSKMNPGEKEPIHRGTTEVNIDSETVHRMLLSAPSENDRVQKNLKNTAAEEHVAQGDATLEHSTNLDSSPSLSSVTVVPLRESYDPDVIPLFDKRTVLEGSTASTSPADHSALPNQSLTVRESEVLKTSDSKEGGEGFTVDTPAKASITSKRHIPEAHQATLLDGKQGKVIMPLGSKLTGVIVENENITKEGGLVDMAKKENDLNAEPNLKQTIKATVENGKKDGIAVDHVVGLNTEKYAETVKLKHKRSPGKVKDISIDVERRNENSEVDTSAGSGSAPSVLHQRNGQTEDVATGPRRAEKTSVATSTEGKDKDVTLSPVKAGPATTTSSETRQSEVALPCTSIEADEGLIIGTHSRNNPLHVGAEASECTVFAAAEEGGAVVTEGFAESETFLTSTKEGESGECAVAESEDRAADLLAVHAVKIEANVNSVVTEEKDDAVTSAGSEEKCDGSLSRDSEIVEGTITFISEVESDGAVTSAGTEIRAGSISSEEVDGSQGNMMRMGPKKETEGTVTCTGAEGRSDNFVICSVTGAGPREERMVTGAGVVLGDNDAPPGTSASQEGDGSVNDGTEGESAVTSTGITEDGEGPASCTGSEDSSEGFAISSESEENGESAMDSTVAKEGTNVPLVAAGPCDDEGIVTSTGAKEEDEEGEDVVTSTGRGNEIGHASTCTGLGEESEGVLICESAEGDSQIGTVVEHVEAEAGAAIMNANENNVDSMSGTEKGSKDTDICSSAKGIVESSVTSAVSGKDEVTPVPGGCEGPMTSAASDQSDSQLEKVEDTTISTGLVGGSYDVLVSGEVPECEVAHTSPSEKEDEDIITSVENEECDGLMATTASGDITNQNSLAGGKNQGKVLIISTSTTNDYTPQVSAITDVEGGLSDALRTEENMEGTRVTTEEFEAPMPSAVSGDDSQLTASRSEEKDECAMISTSIGEEFELPISSATTIKCAESLQPVAAAVEERATGPVLISTADFEGPMPSAPPEAESPLASTSKEEKDECALISTSIAEECEASVSGVVVESENERAGTVMEEKDGSGIISTSSVEDCEGPVSSAVPQEEGDPSVTPAEEMGDTAMISTSTSEGCEAVMIGAVLQDEDRLTITRVEDLSDAAIISTSTAECMPISASIDRHEENQLTADNPEGNGDLSATEVSKHKVPMPSLIAENNCRCPGPVRGGKEPGPVLAVSTEEGHNGPSVHKPSAGQGHPSAVCAEKEEKHGKECPEIGPFAGRGQKESTLHLINAEEKNVLLNSLQKEDKSPETGTAGGSSTASYSAGRGLEGNANSPAHLRGPEQTSGQTAKDPSVSIRYLAAVNTGAIKADDMPPVQGTVAEHSFLPAEQQGSEDNLKTSTTKCITGQESKIAPSHTMIPPATYSVALLAPKCEQDLTIKNDYSGKWTDQASAEKTGDDNSTRKSFPEEGDIMVTVSSEENVCDIGNEESPLNVLGGLKLKANLKMEAYVPSEEEKNGEILAPPESLCGGKPSGIAELQREPLLVNESLNVENSGFRTNEEIHSESYNKGEISSGRKDNAEAISGHSVEADPKEVEEEERHMPKRKRKQHYLSSEDEPDDNPDVLDSRIETAQRQCPETEPHDTKEENSRDLEELPKTSSETNSTTSRVMEEKDEYSSSETTGEKPEQNDDDTIKSQEEDQPIIIKRKRGRPRKYPVETTLKMKDDSKTDTGIVTVEQSPSSSKLKVMQTDESNKETANLQERSISNDDGEEKIVTSVRRRGRKPKRSLTVSDDAESSEPERKRQKSVSDPVEDKKEQESDEEEEEEEEDEPSGATTRSTTRSEAQRSKTQLSPSIKRKREVSPPGARTRGQQRVEEAPVKKAKR

SEQ ID NO: 129 - protein, 183 aa, human
MAHGGPRGQLLGADGQEVQPEALMQELSRCQVLQGRPKIFLLQACRGGNRDAGVGPTALPWYWSWLRAPPSVPSHADVLQIYAEAQGSSCRGTPPGSSDQADILTVYSAAEGYVAYRDDKGSDFIQTLVEVLRANPGRDLLELLTEVNRRVCEQEVLGPDCDELRKACLEIRSSLRRRLCLQA

SEQ ID NO: 130 - protein, 84 aa, human
MSYYGSYYRGLGYGCGGFGGLGYGYGCGCGSFRRLGYGCGFGGNGYGYCRPSCYGGYGFSILLKSYPEDTISEVIRRSFNLTKY

SEQ ID NO: 131 - protein, 148 aa, human
MPGGDTTPEEAAAPSCAGYNPGLLLFRAQKAQGACVTSTEGAWPRRASALYGGRKMRCGESGAGPDPRSNSAEVSSSQPALASKSQSKWGPTSNNPRGALTTTEFEMAGNRSQNIKHKQTALIAIPMSSQTPRMLGRPRNQGQLYPQP

SEQ ID NO: 132 - protein, 293 aa, human
MASNVTNKMDPHSMNSRVFIGNLNTLVVKKSDVEAIFSKYGKIAGCSVHKGFAFVQYDKEKNARAAVAGEDGRMIASQVVDINLAAEPKVNRGNAGVKRSAAEMYGSSFDLDYGFQRDYYDGMYSFPARVPPPPPIALAVVPSKRQRLSGNTSRRGKSGFNSKSGKRGSSKSGKLKGDDLQAIKQELTQIKQKVDSLLENLEKIEKEQSKQEVEVKNAKSEEEQSSSSMKKDETHVKMESEGGAEDSAEEGDPLDDDVNEDQGDDQLELIKDDEKEAEEGEDDRDSTNGQDDS

SEQ ID NO: 133 - protein, 244 aa, human
MGDLFSLFWEVDPPPIPVNCAIPNQDYECWKDDSCGTIGSFLLWYFVIVFVLMFFSRASVWMSEDKKDEGSGTSTSVRKASKETSCKRQSKDSAWDPSQTMKKPKQNQLTPVTNSEVALVNAYPEQRRARRQSQFNEVNQNQHDSDTTEYGSEESNSEASSWKESESEHHPSPDSIKRRKMAQRQRNLGSYQMSERHCLHCKALRTNEWLAHHSRQKPSVTPPMKRDSQEESSISDINKKFSKF

SEQ ID NO: 134 - protein, 136 aa, human
MNCDALLHHSAIPEDFLHIFLLLQKISVSLPLSLSQSVCLFYSISLCVSLLLHISLCVSVYVSLSLSSFPCFSLTHTHTHSQLSKDTSVLTFTFCFKQHTHFTLNYTSHAHELSAPSVHPTCVFTFKAAPSPRPAT

SEQ ID NO: 135 - protein, 148 aa, human
MGSQGSGGVPLVQAPYTVLLLPLGTSRQDPGAQSFFLWLRRMQALEREQDALWQGLELLQHGQAWFEDHLREAQRQQLHLGALGENFLTDLHSEPGRPPLAQIQKVNICLQNLIHEKELSRQQKGVTQPKEEMAQRGCTKGPRGPTRV

SEQ ID NO: 136 - protein, 309 aa, human
MKRKNFTEVSEFIFLGFSSFGKHQITLFVVFLTVYILTLVANIIIVTIICIDHHLHTPMYFFLSMLASSETVYTLVIVPRMLLSLIFHNQPISLAGCATQMFFFVILATNNCFLLTAMGYDRYVAICRPLRYTVIMSKGLCAQLVCGSFGIGLTMAVLHVTAMFNLPFCGTVVDHFFCDIYPVMKLSCIDTTINEIINYGVSSFVIFVPIGLIFISYVLVISSILQIASAEGRKKTFATCVSHLTVVIVHCGCASIAYLKPKSESSIEKDLVLSVTYTIITPLLNPVVYSLRNKEVKDALCRVVGRNIS

SEQ ID NO: 137 - protein, 123 aa, human
MNGSNMANTSPSVKSKEDQGLSGHDEKENPFAEYMWMENEEDFNRQVEEELQEQDFLDRCFQEMLDEEDQDWFIPSRDLPQAMGQLQQQLNGLSVSEGHDSEDILSKSNLNPDAKEFIPGEKY

SEQ ID NO: 138 - protein, 73 aa, human
MQFADWLHPSGWTIEILNAYGMGDRKRTNSMSKEAFTPEQLHLEKELGEMRLRPTVLHSQTDHQGFRPIPMMQ

SEQ ID NO: 139 - protein, 72 aa, human
MNYYGNYYGGLGYGYGGFDDLGYGYGCGCGSFRRLGYGGGYGGYGYGSGFGGYGYRSCRPSCYGGYGFSGFY

SEQ ID NO: 140 - protein, 321 aa, human
MHGRAYLLLHRDFCDLKENNYKGITAKPVSEDMMEWEVEIEGLQNSVWQGLVFQLTIHFTSEYNYAPPVVKFITIPFHPNVDPHTGQPCIDFLDNPEKWNTNYTLSSILLALQVMLSNPVLENPVNLEAARILVKDESLYRTILRLFNRPLQMKDDSQELPKDPRKCIRPIKTTSFSDYYQTWSRIATSKATEYYRTPLLKVPNFIGQYYKWKKMDLQHQKEWNLKYSVIKCWLARKRMPHEVTHSMEEIKLCPTLIPTTDEIFLESPTAINSITDIYETEEEGWKSDTSLYENDTDEPREEEVEDLISWTNTLNTNTSED

SEQ ID NO: 141 - protein, 62 aa, human
MGELAASANHGHSPCYPERKGTPGDLSKRKMLVHFYPRRHSHPRATQQWILKNKTLCRRIKE

SEQ ID NO: 142 - protein, 188 aa, human
MFSCCFPTSRGCCFRNGGSESLFRRCRRRLIPHPRRLSPVVIRRIQVPQDSLGQALAGQATPEIPLGLQLHTVLVQEIQELIEAQTLAPGPCAEVRALPAPAAEPEPAWEEAPPERALELEGAPAKDQTNEELPEITEVPESIKRRLGRRVPAATPAPRGNLLLQAWMRVHSWASRLFAPNVLPGTGP

SEQ ID NO: 143 - protein, 262 aa, human
MTQPVPRLSVPAALALGSAALGAAFATGLFLGRRCPPWRGRREQCLLPPEDSRLWQYLLSRSMREHPALRSLRLLTLEQPQGDSMMTCEQAQLLANLARLIQAKKALDLGTFTGYSALALALALPADGRVVTCEVDAQPPELGRPLWRQAEAEHKIDLRLKPALETLDELLAAGEAGTFDVAVVDADKENCSAYYERCLQLLRPGGILAVLRVLWRGKVLQPPKGDVAAECVRNLNERIRRDVRVYISLLPLGDGLTLAFKI

SEQ ID NO: 144 - protein, 344 aa, human
MEAARPFAREWRAQSLPLAVGGVLKLRLCELWLLLLGSSLNARFLPDEEDVDFINEYVNLHNELRGDVIPRGSNLRFMTWDVALSRTARAWGKKCLFTHNIYLQDVQMVHPKFYGIGENMWVGPENEFTASIAIRSWHAEKKMYNFENGSCSGDCSNYIQLVWDHSYKVGCAVTPCSKIGHIIHAAIFICNYAPGGTLTRRPYEPGIFCTRCGRRDKCTDFLCSNADRDQATYYRFWYPKWEMPRPVVCDPLCTFILLLRILCFILCVITVLIVQSQFPNILLEQQMIFTPEESEAGNEEEEKEEEKKEKEEMEMEIMEMEEEKEEREEEEEETQKEKMEEEEK

SEQ ID NO: 145 - protein, 2896 aa, human
MSEKSGQSTKAKDGKKYATLSLFNTYKGKSLETQKTTARHGLQSLGKVGISRRMPPPANLPSLKAENKGNDPNVNIVPKDGTGWASKQEQHEEEKTPEVPPAQPKPGVAAPPEVAPAPKSWASNKQGGQGDGIQVNSQFQQEFPSLQAAGDQEKKEKETNDDNYGPGPSLRPPNVACWRDGGKAAGSPSSSDQDEKLPGQDESTAGTSEQNDILKVVEKRIACGPPQAKLNGQQAALASQYRAMMPPYMFQQYPRMTYPPLHGPMRFPPSLSETNKGLRGRGPPPSWASEPERPSILSASELKELDKFDNLDAEADEGWAGAQMEVDYTEQLNFSDDDEQGSNSPKENNSEDQGSKASENNENKKETDEVSNTKSSSQIPAQPSVAKVPYGKGPSFNQERGTSSHLPPPPKLLAQQHPPPDRQAVPGRPGPFPSKQQVADEDEIWKQRRRQQSEISAAVERARKRREEEERRMEEQRKAACAEKLKRLDEKLGILEKQPSPEEIREREREKEREREKELEKEQEQEREKEREKDRERQQEKEKELEKEQEKQREMEKERKQEKEKELERQKEKEKELQKMKEQEKECELEKEREKLEEKIEPREPNLEPMVEKQESENSCNKEEEPVFTRQDSNRSEKEATPVVHETEPESGSQPRPAVLSGYFKQFQKSLPPRFQRQQEQMKQQQWQQQQQQGVLPQTVPSQPSSSTVPPPPHRPLYQPMQPHPQHLASMGFDPRWLMMQSYMDPRMMSGRPAMDIPPIHPGMIPPKPLMRRDQMEGSPNSSESFEHIARSARDHAISLSEPRMLWGSDPYPHAEPQQATTPKATEEPEDVRSEAALDQEQITAAYSVEHNQLEAHPKADFIRESSEAQVQKFLSRSVEDVRPHHTDANNQSACFEAPDQKTLSAPQEERISAVESQPSRKRSVSHGSNHTQKPDEQRSEPSAGIPKVTSRCIDSKEPIERPEEKPKKEGFIRSSEGPKPEKVYKSKSETRWGPRPSSNRREEVNDRPVRRSGPIKKPVLRDMKEEREQRKEKEGEKAEKVTEKVVVKPEKTEKKDLPPPPPPPQPPAPIQPQSVPPPIQPEAEKFPSTETATLAQKPSQDTEKPLEPVSTVQVEPAVKTVNQQTMAAPVVKEEKQPEKVISKDLVIERPRPDSRPAVKKESTLPPRTYWKEARERDWFPDQGYRGRGRGEYYSRGRSYRGSYGGRGRGGRGHTRDYPQYRDNKPRAEHIPSGPLRQREESETRSESSDFEVVPKRRRQRGSETDTDSEIHESASDKDSLSKGKLPKREERPENKKPVKPHSSFKPDNHVRIDNRLLEKPYVRDDDKAKPGFLPKGEPTRRGRGGTFRRGGRDPGGRPSRPSTLRRPAYRDNQWNPRQSEVPKPEDGEPPRRHEQFIPIAADKRPPKFERKFDPARERPRRQRPTRPPRQDKPPRFRRLREREAASKSNEVVAVPTNGTVNNVAQEPVNTLGDISGNKTPDLSNQNSSDQANEEWETASESSDFNERRERDEKKNADLNAQTVVKVGENVLPPKREIAKRSFSSQRPVDRQNRRGNNGPPKSGRNFSGPRNERRSGPPSKSGKRGPFDDQPAGTTGVDLINGSSAHHQEGVPNGTGQKNSKDSTGKKREDPKPGPKKPKEKVDALSQFDLNNYASVVIIDDHPEVTVIEDPQSNLNDDGFTEVVSKKQQKRLQDEERRKKEEQVIQVWNKKNANEKGRSQTSKLPPRFAKKQATGIQQAQSSASVPPLASAPLPPSTSASVPASTSAPLPATLTPVPASTSAPVPASTLAPVLASTSAPVPASPLAPVSASASVSASVPASTSAAAITSSSAPASAPAPTPILASVSTPASVTILASASIPILASALASTSAPTPAPAASSPAAPVITAPTIPASAPTASVPLAPASASAPAPAPTPVSAPNPAPPAPAQTQAQTHKPVQNPLQTTSQSSKQPPPSIRLPSAQTPNGTDYVASGKSIQTPQSHGTLTAELWDNKVAPPAVLNDISKKLGPISPPQPPSVSAWNKPLTSFGSAPSSEGAKNGQESGLEIGTDTIQFGAPASNGNENEVVPVLSEKSADKIPEPKEQRQKQPRAGPIKAQKLPDLSPVENKEHKPGPIGKERSLKNRKVKDAQQVEPEGQEKPSPATVRSTDPVTTKETKAVSEMSTEIGTMISVSSAEYGTNAKESVTDYTTPSSSLPNTVATNNTKMEDTLVNNVPLPNTLPLPKRETIQQSSSLTSVPPTTFSLTFKMESARKAWENSPNVREKGSPVTSTAPPIATGVSSSASGPSTANYNSFSSASMPQIPVASVTPTASLSGAGTYTTSSLSTKSTTTSDPPNICKVKPQQLQTSSLPSASHFSQLSCMPSLIAQQQQNPQVYVSQSAAAQIPAFYMDTSHLFNTQHARLAPPSLAQQQGFQPGLSQPTSVQQIPIPIYAPLQGQHQAQLSLGAGPAVSQAQELFSSSLQPYRSQPAFMQSSLSQPSVVLSGTAIHNFPTVQHQELAKAQSGLAFQQTSNTQPIPILYEHQLGQASGLGGSQLIDTHLLQARANLTQASNLYSGQVQQPGQTNFYNTAQSPSALQQVTVPLPASQLSLPNFGSTGQPLIALPQTLQPPLQHTTPQAQAQSLSRPAQVSQPFRGLIPAGTQHSMIATTGKMSEMELKAFGSGIDIKPGTPPIAGRSTTPTSSPFRATSTSPNSQSSKMNSIVYQKQFQSAPATVRMTQPFPTQFAPQILSQPNLVPPLVRAPHTNTFPAPVQRPPMALASQMPPPLTTGLMSHARLPHVARGPCGSLSGVRGNQAQAALKAEQDMKAKQRAEVLQSTQRFFSEQQQSKQIGGGKAQKVDSDSSKPPETLTDPPGVCQEKVEEKPPPAPSIATKPVRTGPIKPQAIKTEETKS

SEQ ID NO: 146 - protein, 108 aa, human
DIQMTQSPSPLSASVGDSVTITCQASQDIRNSLIWYQQKPGKAPKFLIYDAENLEIGVPSRFRGSGSGTDFALSISSLQPEDFATYYCQQYYNLPYTFGQGTKLEIKR

SEQ ID NO: 147 - protein, 175 aa, human
MSYNCCSGNFSSRSCGDYLRYPASSRGFSYPSNLVYSTDLCSPSTCQLGSSLYRGCQEICWEPTSCQTSYVESSPCQTSCYRPRTSLLCSPCKTTYSGSLGFGSSSCRSLGYGSRSCYSVGCGSSGVRSLGYGSCGFPSLGYGSGFCRPTYLASRSCQSPCYRPAYGSTFCRSTC

SEQ ID NO: 148 - protein, 101 aa, human
MNKLGHNELKECLKTATDSLQTVQPSISQTCTSYGPALGAPLPGRNEVALLTSLPPNYEISEGKPRAISAYVRAGKGNVTRRRKKTHLGNDDGKKEAQEKM